SEQUENCE LISTING (10 sequences)

SEQ ID NO: 1 - protein, 19 aa, Artificial Sequence
  linker sequence for VL and VH domains, of the anti-CD19 scFv.
GGGGSGGGGSGGGGSGGGG

SEQ ID NO: 2 - protein, 17 aa, Artificial Sequence
  linker sequence of VL and VH domains, of the anti-AF20 scFv.
ADTTPKLEEGEFSEARV

SEQ ID NO: 3 - DNA, 771 bp, Artificial Sequence
  Assembled M18 anti-protective antigen scFv sequence.
cccctgcagatggctgactacaaagacatccagatgacccagaccacctcttctctgtctgcttctctgggtgaccgtgttaccgtttcttgccgtgcttctcaggacatccgtaactacctgaactggtaccagcagaaaccggacggtaccgttaaattcctgatctactacacctctcgtctgcaaccgggtgttccgtctcgtttctctggttctggttctggtaccgactactctctgaccatcaacaacctggaacaggaagacatcggtacctacttctgccagcagggtaacaccccgccgtggaccttcggtggtggtaccaaactggaaatcaaacgtggtggaggcgggtcaggcggaggtggctccggaggtggcggatcgggtggcggagggtctgaagttcagctgcaacagtctggtccagaactggttaaaccgggtgcttctgttaaaatctcttgcaaagactctggttacgctttcaactcttcttggatgaactgggttaaacagcgtccgggtcagggtctggaatggatcggtcgtatctacccgggtgacggtgactctaactacaacggtaaattcgaaggtaaagctatcctgaccgctgacaaatcttcttctaccgcttacatgcagctgtcttctctgacctctgttgactctgctgtttacttctgcgctcgttctggtctgctgcgttacgctatggactactggggtcagggtacctctgttaccgtttcttcttaagga

SEQ ID NO: 4 - DNA, 759 bp, Artificial Sequence
  Assembled anti-AF20 scFv sequence.
ctgcagggcggcggccagctccagcagtctggtccggacctggttaaaccgggtgcttctgttcgtatctcttgcaaggcttctggttacaccttcgctggtcactacgttcactgggttaaacagcgtccgggtcgtggtctggaatggatcggttggatcttcccgggtaaagttaacaccaaatacaacgaaaaattcaaaggtaaagctaccctgaccgctgacaaatcttcttctaccgcttacatgcagctgtcttctctgacctctgaagactctgctgtttacttctgcgctcgtgttggttacgactacccgtactacttcgactactggggtcagggtaccaccctgaccgtttcttctggaggtggcgggtctgggggcggtggatcgggcggtggaggatcaggcggaggtgggtccgacatcctgctgacccagtctccggctatcctgtctgtttctccgggtgaccgtgtaagcttctcttgccgtgcttctcagtctatcggtacctctatccactggtaccagcagcgtaccaacggttctccgcgtctgctgatcaaatacgcttctgaatctatctctggtatcccgtctcgtttctctggttctggttctggtaccgacttcaccctgtctatcaactctgttgaatctgaagacgttgctgactactactgccagcagtcttcttcttggccgttcaccttcggttctggtaccaaactggaaatcaaataaggatcc

SEQ ID NO: 5 - DNA, 783 bp, Artificial Sequence
  Assembled anti-CD19 scFv sequence.
ctgcagggcggcggccaggttcagctgcaacagtctggtgctgaactggttcgtccgggttcttctgttaaaatctcttgcaaagcttctggttacgctttctcttcttactggatgaactgggttaaacagcgtccgggtcagggtctggaatggatcggtcagatctggccgggtgacggtgacaccaactacaacggtaaattcaaaggtaaagctaccctgaccgctgacgaatcttcttctaccgcttacatgcagctgtcttctctggcttctgaagactctgctgtttacttctgcgctcgtcgtgaaaccaccaccgttggtcgttactactacgctatggactactggggtcagggtacctctgttaccgtttcttctgctgacaccaccccgaaactggaagaaggtgaattctctgaagctcgtgttgacatcctgctgacccagaccccggcttctctggctgtttctctgggtcagcgtgctaccatctcttgcaaagcttctcagtctgttgactacgacggtgactcttacctgaactggtaccagcagatcccgggtcagccgccgaaactgctgatctacgacgcttctaacctggtttctggtatcccgccgcgtttctctggttctggttctggtaccgacttcaccctgaacatccacccggttgaaaaagttgacgctgctacctaccactgccagcagtctaccgaagacccgtggaccttcggtggtggtaccaaactggaaatcaaacgttgaggatcc

SEQ ID NO: 6 - protein, 23 aa, Artificial Sequence
  Histidine rich fusogenic peptide H5WYG of coat protein.
GLFHAIAHFIHGGWHGLIHGWYG

SEQ ID NO: 7 - protein, 38 aa, Artificial Sequence
  nuclear localization sequence derived from the M9 domain of the heterogenous nuclear ribonucleoprotein A1.
NQSSNFGPMKGGNFGGRSSGPYGGGGQYFAKPRNQGGY

SEQ ID NO: 8 - RNA, 21 nt, Bacteriophage MS2
aaacaugaggauuacccaugu

SEQ ID NO: 9 - DNA, 4486 bp, Artificial Sequence
  pDSP62
ttcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcttttcaaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagtttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggcaagcaatttcacgcaatttgtattggtagataacgggggtacgggggatgttacggtagcaccttcaaattttgcaaatggtgtagcagagtggatatcaagcaatagcagaagccaagcatataaggttacgtgctcagtaagacaatcaagcgctcaaaacagaaagtatacgataaaggtagaagttccgaaggttgctacgcaaacggtaggtggtgttgaattgccggttgcagcttggagaagctatctcaacatggagttgacgatacctatatttgcaaccaacagtgattgtgaattgatagtaaaagctatgcaggggttgttgaaggacggtaatcctataccgagcgctatagctgctaatagtggcctctacggcaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccgggttaattaattaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgaa

SEQ ID NO: 10 - DNA, 4498 bp, Artificial Sequence
  pDSP62ampst
ttcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcttttcaaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagtttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggcaagcaatttcacgcaatttgtattggtagataacgggggtacgggggatgttacggtagcaccttcaaattttgcaaatggtgtagcagagtggatatcaagcaatagcagaagccaagcatataaggttacgtgctcagtaagacaatcaagcgctcaaaacagaaagtatacgataaaggtagaagttccgaaggttgctacgcaaacggtaggtggtgttgaattgccggttgcagcttggagaagctatctcaacatggagttgacgatacctatatttgcaaccaacagtgattgtgaattgatagtaaaagctatgcaggggttgttgaaggacggtaatcctataccgagcgctatagctgctaatagtggcctctacggcaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactagggcggcggcctgcagacgccgggttaattaattaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgaa

(19) United States
(12) Patent Application Publication
(10) Pub. No.: US 2013/0017210 A1
(43) Pub. Date: Jan. 17, 2013

(54) DISPLAY OF ANTIBODY FRAGMENTS ON VIRUS-LIKE PARTICLES OF RNA BACTERIOPHAGES

(71) Applicants: David S. Peabody, Albuquerque, NM (US); Bryce Chackerian, Albuquerque, NM (US)

(72) Inventors: David S. Peabody, Albuquerque, NM (US); Bryce Chackerian, Albuquerque, NM (US)

(73) Assignee: STC.UNM, Albuquerque, NM (US)

(21) Appl. No.: 13/583,047

(22) PCT Filed: Mar. 17, 2011
(86) PCT No.: PCT/US11/28875

§ 371 Date: Sep. 6, 2012

Related U.S. Application Data
(60) Provisional application No. 61/314,625, filed on Mar. 17, 2010.

(51) Int. Cl. A61K 39/395; A61P 31/12; A61P 35/02; C12N 1/21; C12N 15/63; C12N 15/11; C40B 50/06; C40B 30/04; A61P 35/00; G01N 33/574

(52) U.S. Cl. 424/178.1; 435/7.23; 435/320.1; 536/23.1; 506/26; 506/9; 435/252.33; 435/252.34

(57) ABSTRACT

The invention enables the display of antibody single-chain variable fragments (scFv's on virus-like particles (VLPs) of bacteriophages such as MS2. The VLPs encapsidate mRNA encoding the coat protein from which it assembles, enabling the recovery by reverse transcription and PGR of affinity-selected sequences from scFv libraries. Related virus-like particles, method for constructing a library of scFv-VLPs, drug delivery vehicles comprising one or more pharmaceutically-active ingredients, biomedical imaging agents, assays, and kits are also provided.

### RELATED APPLICATIONS AND GOVERNMENT INTEREST

This application claims the benefit of priority of provisional application U.S. 61/314,625, filed Mar. 17, 2010 of identical title, the entire contents of which are incorporated by reference herein in its entirety.

This patent application was supported by Grant No. R01 GM042901 from the National Institutes of Health. The Government has certain rights in the invention.

### FIELD OF THE INVENTION

The invention enables the display of antibody single-chain variable fragments (scFv's) on virus-like particles (VLPs) of bacteriophages such as MS2. The VLPs encapsidate mRNA encoding the coat protein from which it assembles, enabling the recovery by reverse transcription and PCR of affinity-selected sequences from scFv libraries.

Related virus-like particles, method for constructing a library of VLPs, immunogenic compositions, drug delivery vehicles comprising one or more pharmaceutically-active ingredients, vaccines comprised of VLPs as described herein, assays, and kits are also provided.

### BACKGROUND OF THE INVENTION

This document describes methods for the display of single-chain antibody variable fragments (scFv's) on the virus-like particles of RNA bacteriophages like MS2. Below are described two possible applications of the invention: 1. Targeted delivery of drugs or imaging agents to specific cell-types mediated by scFv's displayed on VLPs. 2. VLP display of randomized single chain antibody variable fragment (scFv) libraries for affinity selection.

A single-chain antibody variable fragment (or scFv) is an artificial construct that links the sequences encoding the V_{H} and V_{L} domains of an antibody into a single polypeptide chain and lacks the rest of the antibody molecule. Because the antigen-binding site of an antibody is formed in a cavity at the interface between V_{H} and V_{L} domains, the scFv preserves the antigen binding activity of the intact antibody molecule. Normally the V_{H} and V_{L} domains are parts of different polypeptide chains (the heavy and light chains, respectively), but in the scFv they are joined into a single polypeptide that can be fused genetically to other proteins, for example, the coat proteins of RNA bacteriophages like MS2 in the present invention. A schematic summary of antibody structure and the relationship to the scFv is shown in FIG. 1.

1. Targeted Delivery of Drugs or Imaging Agents to Specific Cell-Types Mediated by scFv's Displayed in VLPs.

We have recently described the use of virus-like particles (VLPs) for targeted delivery of drugs to mammalian cells. VLPs were directed to a specific cell type by decorating the VLP with a peptide able to bind a receptor protein present on the cell surface. Subsequent endocytosis of the VLP delivered its molecular cargo (a drug or imaging agent) to the cell interior. Although proof-of-concept experiments were conducted using peptides conjugated chemically to the virus-like particle, the ability to construct peptide libraries directly on MS2 VLPs opens the possibility of performing targeting peptide identification and drug delivery on a single platform.

Monoclonal antibodies offer an alternative to peptides as a means of directing a VLP to bind to a cell surface receptor. In fact, antibodies may be superior to peptides in some respects, as they sometimes exhibit higher affinities and specificities for their targets. Moreover, monoclonal antibody reagents are already available for a huge number of different human cell surface proteins, and many of them could be adapted for targeting purposes. Single-chain variable fragments (scFv's) are produced by recombinant DNA methods and consist of an antibody's two variable domains (VH and VL) joined together through a linker sequence into a single polypeptide chain. They contain the site for antigen binding and therefore possess the ligand recognition properties of the antibody from which they are derived. Filamentous phages have been previously adapted for display of scFv's. In fact libraries of diverse scFv's have been created from which scFv's with specific binding functions have been isolated by affinity selection. This application describes display of scFv's on RNA bacteriophage VLPs.

2. VLP Display of Randomized scFv Libraries for Affinity Selection.

Phage display is one of several technologies that make possible the presentation of large libraries of random amino acid sequences with the purpose of selecting from them peptides with certain specific functions. Phage display is used both for peptide and scFv display, and both are briefly summarized here, although scFv display is the primary focus of this application. The basic idea is to create recombinant bacteriophage genomes containing a library of randomized sequences (typically 7-12 amino acids in length) genetically fused to one of the structural proteins of the virion. When such recombinants are transfected into bacteria each produces virus particles that display a particular peptide on their surface and which package the same recombinant genome that encodes that peptide, thus establishing the linkage of genotype and phenotype essential to the method. Arbitrary functions (e.g. the binding of a receptor) can be selected from such libraries by the use of biopanning and other techniques. In addition to presentation of random peptide sequences, it is also possible to present single-chain antibody fragments on a bacteriophage surface. Libraries can be constructed that reflect the natural diversity of the antibody repertoire from which scFv's with desired specificities can be affinity-selected. Display of scFv's is normally conducted on filamentous phages (e.g. M13). It is a well-developed technology, but relatively heroic efforts have been required in order to construct libraries containing as many as about 10¹⁰ individual members. Most libraries are significantly smaller. The complexities of these libraries are constrained by the need to produce scFv DNA fragments, to ligate them to phage DNA, and then to introduce the ligated DNA into E. coli by transformation. This requirement for passage through E. coli is the result of the relatively complex makeup of the virions of the phages used for phage display, and the consequent necessity that their components be synthesized and assembled in vivo.

### SUMMARY OF THE INVENTION

In one aspect of the present invention, the disclosure provides a system or a method for using virus-like particles as a platform for a random peptide library, wherein the peptide library comprises one or more peptides that selectively bind to an organic, inorganic, or biological material.

In another aspect, the disclosure provides a system or a method for using virus-like particles to encapsidate various cargo components comprising nucleic acids, such as biologically active RNA) including small interfering RNA (siRNA), micro RNA (miRNA) a short hairpin RNA (shRNA) or a mixture thereof) quantum dots, gold nanoparticles, iron oxide nanoparticles, or RNA-modified cargoes for therapeutic and/or imaging applications.

In yet another aspect, the disclosure provides a system or a method for internalizing virus-like particles within a target cell, including a cancer cell, wherein one or more cargo components loaded in the virus-like particles are released upon the internalization within the cell, which in preferred aspects is a cancer cell or infected cell.

An additional embodiment of the present invention is directed to a virus-like particle (VLP) of a bacteriophage comprising an interior core surrounded by a capsid comprising a coat protein of the bacteriophage, wherein the coat protein comprises a scFv polypeptide that specifically binds to a target cell. In one embodiment, the bacteriophage is an RNA bacteriophage selected from the group consisting of MS2, Qβ, R17, SP, PP7, GA, M11, MX1, f4, AP205, PRRI, Cb5, Cb12r, Cb23r, 7s and f2. In certain embodiments, the RNA bacteriophage is MS2 or Qβ. In other embodiments, the capsid of the VLP is optionally PEGylated.

In another embodiment, the target cell is a cancer cell and the scFV polypeptide binds specifically to the cancer cell.

In certain aspects, the interior core of the VIPs described herein optionally comprise one or more cargo components (e.g., one or more bioactive agents, for example, cytotoxic agents, such as a chemotherapeutic or other drug, an RNA molecule, or a toxin, or one or more imaging agents as otherwise in greater detail herein).

In one embodiment the drug, is a chemotherapeutic agent or a mixture of chemotherapeutic agents as otherwise described herein. In another embodiment, the toxin comprises the A chain of the ricin toxin. In another embodiment, the RNA molecule is a biologically active RNA, including a small-interfering RNA (siRNA), micro RNA (miRNA) short hairpin RNA (shRNA) or a mixture thereof. In certain embodiments, the siRNA suppresses the expression of one or more cyclin genes (e.g., cyclin A2, B1, D1, and or E1). The siRNA optionally comprises a nuclear localization sequence that directs the siRNA to the nucleus of the target cell.

In yet another embodiment, the one or more bioactive agents, including a cytotoxic agent or one or more imaging agents are coupled to a nucleic acid molecule, such as a bacteriophage (e.g., MS2 or Qβ) pac site that induces formation of the VLP and encapsidation of the one or more bioactive agent, including a cytotoxic agents or one or more imaging agents within the interior core of the VLP. In certain embodiments, the MS2 pac site comprises the nucleic acid sequence of SEQ ID NO:8 AAACAUGAGGAUUACCCAUGU (See FIG. 15). A generalized structure of the MS2 pac site (a RNA hairpin specifically recognized by the coat protein as defined generically) is presented in FIG. 15 (above right).

The one or more bioactive agent, including a cytotoxic agent are optionally coupled to the nucleic acid molecule via a crosslinker molecule. In certain aspects of the invention, the crosslinker molecule is a cleavable crosslinker molecule.

In still other aspects of the invention, the coat protein comprises a histidine rich fusogenic peptide H5WYG, comprising peptide sequence GLFHAIAHFIHGGWHGLIHGWYG (SEQ ID. NO:6) which upon protonation (pKₐ=6.0), induces osmotic swelling and membrane destabilization of endosomes without affecting the Integrity of the plasma membrane. The histidine-rich fusogenic peptide is optionally synthesized with a C-terminal cysteine residue separated from the histidine-rich fusogenic peptide by a (Gly)₂ spacer.

Another aspect of the invention is directed to a method of treating cancer in a patient, comprising administering to the patient a VLP, as described herein, wherein the interior core of the VLP comprises one or more cytotoxic agents in an amount sufficient to treat the cancer. In one embodiment, the cancer is a cancer for which the scFv specifically binds and the method comprises administering to the patient a VLP that specifically binds to that cancer as described herein, wherein the interior core comprises one or more cytotoxic agents in an amount effective to treat that cancer.

In one embodiment, the one or more cytotoxic agents as otherwise described herein. comprise one or more anticancer agents as specifically disclosed herein, including a mixture of anticancer agents specific for a cancer for which the scFV peptide is also specific. In another embodiment, the one or more cytotoxic agents comprise the A chain of the ricin toxin. In yet another embodiment, the one or more cytotoxic agents comprise an RNA molecule, including, for example, a siRNA. In certain embodiments, the siRNA suppresses the expression of one or more cyclin genes (e.g., cyclin A2, B1, D1, and/or E1). The siRNA optionally comprises a nuclear localization sequence that directs the siRNA to the nucleus of the target cell.

Certain methods are directed to killing a cancer cell in a patient and comprise administering to the patient a VLP, as described herein, wherein the interior core of the VLP comprises one or more cytotoxic agents in an amount sufficient to kill the cancer cell. In one embodiment, the cancer is as otherwise described herein.

Another aspect of the invention is directed to a pharmaceutical composition comprising a VLP, as described herein, and a pharmaceutically acceptable carrier, additive or excipient.

Still another aspect is directed to a method of determining whether a sample contains one or more cancer cells comprising treating the sample with a VLP, as described herein, wherein the interior core of the VLP comprises one or more imaging agents, removing unbound VLP from the sample, and determining if the one or more imaging agents are detected m the sample, wherein if the one or more imaging agents are detected in the sample, the sample contains the cancer cell. In varying aspects of the invention, the cancer is as otherwise described in detail herein.

The present invention enables the display of scFv's on the VLPs of bacteriophages such as MS2 and Qβ, among others. Among the broader applications of this system are the following:

1. Targeted Delivery of Drugs and Imaging Agents to Cells.

VLPs decorated with scFv's specific for cell-surface receptors, may bind and enter cell bearing such receptors. When the interior of the VLP has been loaded with a molecular cargo (e.g. a cytotoxin) that cargo is delivered to the cell. In favorable cases, cells may be killed in a highly specific fashion.

2. Display of scFv Libraries for Affinity Selection.

The VLP encapsidates the mRNA that encodes the coat protein from which it assembles, making possible the recovery by reverse transcription and PCR of affinity selected sequences from scFv libraries.

In the present invention, an scFv is preferably fused to coat proteins, usually at the C-terminus, which may be a single-chain coating protein dimer, or alternatively with wild-type coat protein. The valency of the resulting scFv display optionally may be kept low by the insertion of a stop codon (e.g. an amber codon or as otherwise described herein) between the coat protein and scFv sequences so that the fusion protein is only produced in the presence of a suppressor tRNA. The relative amounts of coat protein (i.e. without scFv) or coat protein-scFv fusion are determined by the efficiency of the nonsense suppression, which can be varied, but which is preferably no more than a few percent (preferably, ranging from about 0.5% to about 5-10%). Alternatively, a direct fusion (i.e., with no intervening stop codon) also produces a properly assembled virus-like particle, but in such case, the scFv is displayed at high valency (i.e. 180 per VLP if fused to the wild-type coat protein, 90 if fused to the single-chain dimer). The examples constructed so far have fused scFv's to coat protein's C-terminus, but N-terminal fusions are also envisioned. Again, the fusions may be constructed wither with or without an intervening stop codon. In the case of an N-terminal fusion with an intervening stop codon, it would be necessary to furnish a ribosome binding sequence for both the upstream scFv sequence (i.e., 5′ to the scFv sequence) and for the coat sequence itself (5′ t the coat sequence), so that the translation of each can be initiated independently. The invention also anticipates the insertion of scFv sequences at an internal location of the coat polypeptide at the top of a β-hairpin in a location known as the AB-loop. FIG. 3 illustrates some of these coat protein-scFv fusion modes.

Direct fusions (i.e. with no intervening stop codon) expressed in E. coli will result in production of the fusion protein only. However, the scFv display valency can be varied by co-assembly in vitro of appropriate mixtures of purified coat protein (e.g. single-chain dimer or wild-type coat protein) and a scFv-coat protein fusion.

In one embodiment the present invention provides plasmids for the production of a coat protein (usually in the form of the single-chain dimer) and the same coat protein fused to the sequence of a single-chain antibody fragment from a single messenger-RNA. In the configuration described here the VLP is formed by the co-assembly of these two proteins into a mosaic capsid. Accordingly, the present invention provides one and optionally, two types of nucleic acid constructs.

A first plasmid which comprises:

(a) a bacterial or bacteriophage promoter which is operably associated with a coding sequence of bacteriophage coat protein or single-chain coat polypeptide dimer,
(b) a nucleotide sequence which encodes an antibody single-chain variable fragment and which is in-frame with, and positioned 3′ to, the coat polypeptide dimer coding sequence's termination codon;
(c) a first restriction site positioned 3′ to the coat polypeptide dimer coding sequence and 5′ to the antibody single-chain variable fragment nucleotide sequence and a second restriction site positioned 3′ to the antibody single-chain variable fragment nucleotide sequence;
(d) a PCR primer positioned 3′ to the second restriction site;
(e) a gene for resistance to a first antibiotic; and
(f) a replication origin for replication in a prokaryotic cell.

And optionally, a second plasmid, in combination with said first plasmid, which comprises:

(a) the gene for a nonsense suppressor tRNA to promote translational readthrough of the coat protein stop codon in said first plasmid, thus allowing the synthesis of a coat protein-scFv fusion protein;
(b) a procaryotic origin of replication, preferably from a different plasmid incompatibility group than that of the first plasmid; and
(c) a gene for resistance to a second antibiotic.

In a preferred embodiment, the first plasmid contains the gene for a coat protein or coat protein single-chain dimer, whose reading frame terminates with a UAG (i.e. amber) stop codon. The UAG separates the coat sequence from a downstream scFv, which is in the same reading frame as the coat sequence. A second plasmid produces an amber-suppressing tRNA, which allows a percentage of ribosomes translating the coat sequence to readthrough into the scFV gene, thus producing a coat protein-scFV fusion protein. The coat protein and coat protein-scFV fusion coassemble into virus-like particles, which encapsidate the coat-scFV mRNA. The valency of scFv display depends on the suppression efficiency. In another embodiment, the stop codon is replaced with a sense codon, so that every coat protein molecule is fused to the scFV. In this embodiment high scFv display valency is achieved, either 180 copies per VLP when fused to coat protein (e.g. wild-type), or 90 copies per VLP when fused to the single-chain coat protein.

In another embodiment mosaic VLPs can be obtained by co-expression of the coat protein and coat protein-scFv fusion from different genes, on the same or different plasmids.

In another embodiment it is anticipated that the two forms of the protein (single-chain coat protein with and without the scFv fusion) will be separately synthesized in bacteria, the two proteins separately purified, and the VLP formed by assembly in vitro from mixtures of the two disaggregated proteins. The coat protein-scFv fusion would be produced without an intervening stop codon. The valency of scFv display depends on the ratio of the two species in the assembly reaction. This method is especially applicable to targeted delivery applications where disassembly-reassembly may be needed for cargo loading. Affinity selection applications, on the other hand, normally require production of both proteins from a single gene so as to provide for encapsidation of the mRNA that encodes the proteins. Plasmids for the synthesis of several different forms of coat proteins and coat-scFv fusions are illustrated in FIGS. 2 and 3.

In another embodiment of the nucleic acid constructs of the invention, the constructs optionally comprise a transcription terminator positioned downstream of the coat protein and scFv coding sequences.

Nucleic acid constructs of the invention are useful in the expression of virus-like particles comprised of a coat polypeptide of a bacteriophages such as MS2 modified by genetic fusion of an antibody single-chain variable fragment, wherein the antibody single-chain variable fragment is displayed on the virus-like particle and encapsidates the bacteriophage mRNA.

In other embodiments, the invention provides virus-like particles made by transforming a prokaryote with antibody single-chain variable fragment sequence-containing constructs as described herein, methods for constructing a library of such virus-like particles, methods for identifying peptides and for isolating an immunogenic protein, immunogenic compositions, drug delivery vehicles which comprise one or more pharmaceutically-active ingredients, vaccines comprising virus-like particles made by transforming a prokaryote with antibody single-chain variable fragment sequence-containing constructs as described herein, and diagnostic assays or kits comprising a virus-like particles made by transforming a prokaryote with antibody single-chain variable fragment sequence-containing constructs as described herein.

For example, in one embodiment, the invention provides a method for constructing a library of virus-like particles, the method comprising:

(a) providing a plurality of a nucleic acid constructs comprising (1) a bacterial or bacteriophage promoter which is operably associated with a coding sequence of a bacteriophage coat protein or single chain coat polypeptide dimer, (2) a first restriction site positioned 3′ to the coat polypeptide dimer coding sequence and 5′ to the antibody single-chain variable fragment nucleotide sequence and a second restriction site positioned 3′ to the antibody single-chain variable fragment nucleotide sequence;
(3) a PCR primer positioned 3′ to the second restriction site (4) a gene for resistance to a first antibiotic (4 a replication origin for replication in a prokaryotic cell.
(b) treating the nucleic acid constructs with a restriction enzyme;
(c) obtaining a population of transcription units by inserting into the nucleic acid constructs, in a position which is in-frame with and 3′ to the coat polypeptide dimer coding sequence's termination codon, a nucleotide sequence which encodes an antibody single-chain variable fragment; and
(d) expressing the transcription units and, optionally, isolating the library (e.g., using biopanning or other technique for separating out the scFv containing coat polypeptide from other biological material including non-scFv containing coat polypeptide), wherein each particle comprises a bacteriophage coat polypeptide modified by insertion of the antibody single-chain variable fragment, and wherein the antibody single-chain variable fragment is displayed on the virus-like particle and encapsidates bacteriophage mRNA.

Methods and constructs of the invention enable the display of antibodies on a bacteriophage VLP, and therefore provide a means of targeting VLPs to cell types that express particular surface proteins. For example, the protein known as CD99 is present in small amounts on the surfaces of many different types of human cells, but it is dramatically over-expressed in certain cancers. Because of its increased concentration on some tumor cells, CD99 is one example of a possible target for cell-specific drug delivery. Other examples in addition to CD99, include CD19, CD22, CRLF2 transferrin receptor and numerous others. In accordance with the instant invention, existing monoclonal antibodies for CD99 (or other targets) are cloned as scFV's and displayed on the VLP. Cells expressing high levels of CD99 would be targeted by VLPs displaying an anti-CD99 scFv. Such particles can be loaded with a variety of bioactive agents such as drugs, especially anti-HIV agents or other biologically active cargo. For example, VLPs can be loaded with anti-tumor drugs like doxorubicin for specific delivery to CD99-expressing cells. Furthermore, VLPs readily encapsidate biologically active RNAs (e.g. small interfering RNAs [siRNA]) that have the ability to interfere with essential activities of the target cell. SiRNAs are especially promising cargo because of the specificity with which they may target an intracellular activity. The combination of extracellular and intracellular targeting specificities should enormously increase the overall specificity for the desired target cell. For example, in some tumor cells the continued expression of a specific gene is required for maintenance of the transformed state. SiRNAs can be designed to specifically target the expression of that gene. More generally, all dividing cells require the expression of cyclins. SiRNAs that inhibit cyclin expression can induce programmed cell death. Another example is the ricin A-chain. Ricin itself consists of two polypeptide chains, called A and B. The A-chain functions as a specific ribonuclease that inactivates the ribosomes of mammalian cells. The B-chain is a lectin-binding protein that binds the cell surface and is endocytosed. The A-chain depends on the B-chain for cell entry and is therefore essentially non-toxic in isolation. When VLPs are loaded with ricin A-chain, their ability to intoxicate cells becomes dependent on the VLP and any targeting molecule (e.g. an scFv) that it displays.

Further, the invention enables the production of scFv libraries that use cells as affinity selection agents to identify new targeting antibodies. This means that scFv targeting of cells is not restricted to the use of previously identified antibodies. The VLP platform can be used for the construction of scFv libraries, from which specific cell-targeting scFv's may be isolated by affinity selection against cellular targets or against purified cell surface receptor molecules.

The invention offers several advantages. It enables the creation of scFv libraries in which each member of a complex phage population expresses a different antibody. Such libraries can be constructed by cDNA cloning of mRNAs extracted from cells of the immune system, thus producing a phage-displayed representation of the entire antibody repertoire. It is also possible to create libraries on the structural framework of a particular antibody by randomizing the sequence of the so-called complementarity-determining regions of its antigen-binding site. From these libraries, it is possible to select specific antibodies by their affinity for a particular antigen (e.g. by biopanning or other approach).

Further, it is technically cumbersome to construct complex scFv libraries on conventional phage display platforms. The potential to construct bacteriophage VLP libraries entirely in vitro in accordance with the invention makes the construction of complex libraries far more convenient. Since the VLP encapsdiates the mRNA that encodes the coat-scFv fusion, affinity selected sequences can be recovered by reverse transcription and PCR.

These and other aspects of the invention are described further in the Detailed Description of the Invention.

### BRIEF DESCRIPTION OF THE FIGURES

FIG. 1 depicts the structure of prototypical antibody molecule (e.g. IgG) and its relationship to the scFv, which is derived from it. A prototypical antibody molecule is comprised of two identical heavy chains and two identical light chains, which associate with one another into the intact tetrameric IgG molecule. The antigen-binding sites reside in a cleft between the variable domains of the heavy and light chains. This means that the antigen binding site can be reconstructed in a recombinant molecule in which the two types of variable domain are artificially joined into a single-chain variable fragment or scFv.

FIG. 2 depicts the structure of pDSP62 plasmid, a representative plasmid. The coat protein it expresses is a so-called single-chain dimmer (see the text). The 5′-half of the single-chain dimer consists of a “codon juggled” sequence, which contains the maximum possible number of silent mutations compared to the wild-type present in the 3′-half. Note that pDSP62 was the plasmid used to represent this aspect of the invention. It is noted that practically any other coat protein expressing plasmid could also be used. The presence of the codon juggled sequence in place of the normal sequence is irrelevant.

FIG. 3A illustrates that the plasmid pDSP62-M18 which was constructed from pDSP62ampst (see FIG. 4) by insertion of the M18 scFv sequence between the PstI and BamHI restriction sites. In cells expressing a suppressor tRNA (e.g. from pNMsupA) this plasmid produces two forms of the coat protein single-chain dimer, with and without the scFv fused to its C-terminus. FIG. 3B shows the design of constructs that fuse the scFv fragment to the N-terminus of coat protein. Two versions of scFv fusions to the N-terminus of the coat protein single-chain dimer. The upper panel illustrates a direct fusion without an intervening stop codon. In this case a single polypeptide chain is produced that contains both scFv and coat sequences. The lower panel shows an arrangement where both scFv-fused and unfused forms of coat protein are produced. The scFv-coat fusion is produced when the stop codon is suppressed by a suppressor tRNA, allowing ribosomes that initiate at the scFv sequence to read through the stop codon and into the coat protein sequence (in the same frame). Expression of coat protein without the N-terminal scFv fusion requires that a ribosome binding site and initiation codon are provided so as to allow independent translation of the coat sequence.

FIG. 4 illustrates that pDSP62 ampst was constructed from pDSP62 by introduction of an amber codon (UAG) in place of the normal coat protein stop codons, and a PstI site at the indicated location.

FIG. 5 illustrates that pNMsupA synthesizes an alanine-inserting, amber suppressing tRNA on a plasmid compatible with members of the pDSP series.

FIG. 6 illustrates that pDSP1 produces a coat protein single-chain dimer. pDSP1(am) differs from it by the introduction of an amber codon (UAG) at the junction of the two halves of the coat sequence. In the presence of pNMsupA it produces large quantities of wild-type coat protein and small amounts of the single-chain dimer by suppression of the UAG.

FIG. 7 depicts Agarose gel electrophoresis of crude cell lysates from strain C41(DE3)/pNMsupA containing the indicated plasmids (in duplicate). The gel was stained with ethidium bromide and visualized under UV illumination.

FIG. 8 depicts SDS-polyacrylamide gel electrophoresis and western blot analysis of purified VLPs. The blot was probed with rabbit anti-MS2 serum.

FIG. 9 depicts SDS gel electrophoresis and western blot of the indicated VLPs. SDS polyacrylamide gel electrophoresis and western blot of VLPs made from the single-chain coat protein (scCP) itself, and from scCP-based VLPs displaying antAF20 or anti CD19 scFv's. The proteins were detected with antibody to MS2 coat protein.

FIG. 10 depicts fluorescence microscopy of Hep3B hepatocellular carcinoma cells and normal hepatocytes. VLPs (green) were labeled with Alexa Fluor 488, cell cytoplasm was stained with Cell Tracker Red CMFDA, and nuclei were visualized by staining with Hoechst 33342. Cultured hepatocellular carcinoma cells (Hep3B) bind VLPs displaying the antiAF20 scFv, but normal hepatocytes do not. VLPs are labeled green, cytoplasm is stained red and nuclei are stained blue.

FIG. 11 depicts FACS analysis of either Jurkat cells (CD19−) or NALM6 cells (CD19+) bound to VLPs labeled with Alexa Fluor 488. VLPs either displayed no scFv, or displayed the anti-protective antigen scFv M18 (as a negative control), or antiCD19 scFv's. Binding to cells depends on the presence of CD19 on the cells and on anti-CD19 scFv on the VLP. FACS analysis shows that fluorescently labeled VLPs displaying antiCD19 bind CD19 positive cells (NALM6), but not CD19 negative cells (Jurkat). Neither cell type is significantly bound by VLPs that display an irrelevant scFv (M18) or that display no scFv at all.

FIG. 12 depicts the synthesis and structure of a VLP displaying an scFv at low valency by a nonsense suppression mechanism. A plasmid like that of FIG. 3 is transcribed to produce a mRNA which is translated by ribosomes to synthesize two proteins. The first is coat protein (or as in this example a single-chain coat protein dimer). The second is produced (usually in smaller amounts) when a suppressor tRNA (expressed, for example, from the plasmid of FIG. 5) causes ribosomes to occasionally read through a stop codon that separates the coat and scFv sequences. The resulting coat protein and coat-scFv fusion co-assemble into a VLP which contains the mRNA encoding the two proteins. This is the mode of synthesis and assembly which is used in producing particles from a complex scFv library for affinity selection.

FIG. 13 depicts the expression of a library of scFv sequences displayed on VLPs, and the affinity-selection of a VLP-scFv with binding activity for a specific target molecule. 1. A library of scFv's is constructed in an expression plasmid (e.g. pDSP1ampst). 2. The library is introduced into E. coli where each transformant produces a VLP displaying an scFv with a different ligand specificity. 3. VLPs are extracted from cells. 4. VLPs are subjected to affinity selection by binding them, for example, to a receptor molecule immobilized on a plastic surface. 5. VLPs that fail to bind are washed away and discarded. 6. VLPs that bind the receptor molecule are eluted. 7. The RNA contained within the bound VLPs is copied into DNA by reverse transcription and then amplified by polymerase chain reaction. The resulting DNA is recloned for production of VLPs, which are used in additional rounds of affinity selection, or, if selection is complete, the selected scFv's are characterized with respect to binding affinity and specificity and their sequences are determined.

FIG. 14 depicts the assembly in vitro of coat protein and a coat-scFv fusion protein with a specific cargo molecule. When the cargo is not an RNA, it is first modified by chemical attachment of a pac site RNA like that depicted in FIG. 15. This causes the cargo to be incorporated into the assembling VLP. When the cargo is itself an RNA molecule (e.g. siRNA) modification with pac site RNA is unnecessary, although it may be desirable in certain cases. When the VLP-scFv binds an appropriate receptor on a target cell, it is internalized (usually by endocytosis), the cargo molecules are released, and (if the cargo is a cytotoxin) the cell is killed. On the left is illustrated the co-assembly in vitro of coat protein (CP) with the coat protein-scFv fusion (CP-scFv), and the cargo molecule (e.g. a cytotoxin like ricin A-chain) linked to a small RNA that serves as an encapsidation signal. In this case, the two proteins are produced separately, purified, and added to an in vitro assembly reaction with the other components. When the scFv binds a receptor on a target cell (right), the VLP enters (usually by endocytosis) and delivers its cytotoxic cargo to the cell interior, causing the cell to die.

FIG. 15 depicts the structure of the MS2 pac site, an RNA that can stimulate the assembly of the VLP in vitro and mediates the encapsidation of any attached cargo molecules. The structure of the MS2 pac site, an RNA hairpin specifically recognized by coat protein is presented. Attachment of the pac site to cargo molecules by chemical cross-linking allows them to be incorporated into assembled VLPs. At left is the sequence encountered in the MS2 genome. At right is shown the generalized structure of the pac site. A wide variety of nucleotide sequences can satisfy the requirements for coat protein binding, as long as the indicated sequence and base-pairing patterns are preserved. Variants that bind coat protein more tightly than wild-type are known.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient”, “host” or “subject” is used throughout the specification within context to describe an animal, generally a mammal and preferably a human, to whom treatment, including prophylactic treatment (prophylaxis), with the therapeutic, diagnostic and/or immunogenic VLPs compositions and/or vaccines according to the present invention is provided. For treatment or diagnosis of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe a number of VLP's or an amount of a VLP-containing composition or other component of the present invention which, in context, is used to produce or effect an intended result, whether that result relates to the diagnosis or therapy of a given disease state or condition, or the prophylaxis and/or therapy of a disease state and/or condition as otherwise described herein or otherwise, in the methods which are described herein. The term effective subsumes all other effective amount or effective concentration terms (including the term “therapeutically effective”) which are otherwise described or used in the present application.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

Restriction endonucleases are enzymes that cleave DNA at well-defined sequences. They are used in recombinant DNA technology, for example, to generate specific DNA fragments that are readily joined through the action of DNA ligase to other DNA fragments generated by digestion with the same restriction endonuclease. In this application, reference is made to several specific restriction endonucleases, including SalI, KpnI, and BamHI whose recognition sequences are, respectively: GTCGAC (SEQ ID NO:11), GGTACC (SEQ ID NO:12), and GGATCC (SEQ ID NO:13).

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

The term “capsid” refers to the protein shell of a virus, such as a bacteriophage, comprising one or more coat proteins.

The terms “cargo” or “cargo component” include, but are not limited to, cytotoxic agents, proteins, peptides, antibodies, nucleic acids (e.g., DNA or RNA), or imaging agents as otherwise described herein. In embodiments, the cargo components can be loaded in an amount of about 1% to about 50%, by weight or by mole of the porous particle pore, although other loading percentages can also be used and varied.

The term “cytotoxic agent” refers to a substance that inhibits or prevents the function of cells and/or causes destruction of cells. The term is intended to include radioactive isotopes (e.g., ²¹¹At, ¹³¹I, ¹²⁵I, ³²P, ³⁵S and radioactive isotopes of Lu, including ¹⁷⁷Lu, ⁸⁶Y, ⁹⁰Y, ¹¹¹In, ¹⁷⁷Lu, ²²⁵Ac, ²¹²Bi, ²¹³Bi, ⁶⁶Ga, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁴⁸V, ²⁰³Pb, ²⁰⁹Pb, ²¹²Pb, ¹⁶⁶Ho, ¹⁴⁹Pm, ¹⁵³Sm, ²⁰¹Tl, ¹⁸⁸Re, ¹⁸⁶Re and ^{99m}Tc), anticancer agents as otherwise described herein, including chemotherapeutic (anticancer drugs e.g. methotrexate, adriamicin, vinca alkaloids (vincristine, vinblastine, etoposide), taxol, doxoruicin, cisplatin, 5-fluorouridine, melphalan, ethidium bromide, mitomycin C, chlorambucil, daunorubicin and other intercalating agents, enzymes and fragments thereof such as nucleolytic enzymes, antibiotics, therapeutic RNA molecules (e.g., siRNA, antisense oligonucleotides, microRNA, ribozymes, RNA decoys, aptamers) and toxins such as small molecule toxins or enzymatically active toxins of bacterial, fungal, plant or animal origin, such as pokeweed antiviral protein (PAP), ricin toxin A, abrin, gelonin, saporin, cholera toxin A, diphtheria toxin, Pseudomonas exotoxin, and alpha-sarcin, including fragments and/or variants thereof.

The term “imaging agent” refers to any detectable moiety that is capable of producing, either directly or indirectly, a detectable signal. For example, the agent may be a biotin label, an enzyme label (e.g., luciferase, alkaline phosphatase, beta-galactosidase and horseradish peroxidase), a radiolabel (e.g., 3H, 14C, 32P, 35S and 125I), a fluorophore such as a fluorescent or chemiluminescent compound (e.g., fluorescein isothiocyanate, rhodamine), inorganic nanoparticles (e.g., gold particles, magnetic nanoparticles, or quantum dots), and a metal ion (e.g., gallium and europium).

The term “LC90” (alternatively, LD90) refers to lethal concentration or lethal dose 90% or the concentration (dose) of a substance that kills 90% of a population of cells, in particular cancer cells.

The term “MS2 pac site” refers to an RNA molecule that interacts with MS2 coat protein, to induce encapsidation, of the M2 genome during bacteriophage replication. The MS2 pac site (MS2 translational operator), is a 19-nucleotide RNA stem-loop (SEQ ID NO:8) that via its interaction with coat protein, mediates exclusive encapsidation of the MS2 genome during bacteriophage replication. See, for example, Wu, et al., Bioconjugate Chemistry, 6(5):587-595 (1995); Picket and Peabody, Nucl. Acids Res. 21(19):4621-4626 (1993) and Uhlenbeck, O., Nature Structural Biology, 5(3):174-176 (1998). The MS2 operator or pac site, can facilitate efficient encapsidation of non-genomic materials, such as the A-chain of ricin, an anticancer agent, etc., within the interior volume of the MS2 VLPs upon conjugation of the pac site to the cargo of interest. See, Wu, et al., Bioconjugate Chemistry, 6(5):587-595 (1995) and Wu, et al., Nanomedicine: Nanotechnology, Biology and Medicine 1(1):67-76 (2005). MS2 VLPs will also encapsidate RNA hairpins with sequences that differ from that of the native operator (Uhlenbeck, O., Nature Structural Biology, 5(3):174-176 (1998)) as well as heterologous nucleic acids, including single and double-stranded RNA and DNA less than about 3 kbp in length. Accordingly, the sequence of the pac site may be modified as long as the modification does not prevent the RNA molecule from inducing VLP self assembly. For example, the pac site can further comprise a spacer molecule, for example, a polyU or oligoU nucleotide (e.g., (U)₃₋₉).

The term “specific binding” or “specifically binds” refers to two molecules forming a complex that is relatively stable under physiological conditions. Specific binding is characterized by a high affinity and a low to moderate capacity as distinguished from nonspecific binding which usually has a low affinity with a moderate to high capacity. Typically, binding is considered specific when the difference between the dissociation constant (K_{d}) for a target cell is at least 1000-fold less than the K_{d} for a control, non-target cell. If necessary, nonspecific binding can be reduced without substantially affecting specific binding by varying the binding conditions.

The terms “treating” or “treatment” refer to any treatment of any disease or condition in a mammal e.g. particularly a human, a mouse, rat or domesticated animal, and includes inhibiting a disease e.g., cancer), condition, or symptom of a disease or condition, e.g., arresting its development and/or delaying its onset or manifestation in the patient or relieving a disease, condition, or symptom of a disease or condition, e.g., causing regression of the condition or disease and/or its symptoms.

The term “isolated” is used within the context of a biological molecule to refer to a biological molecule which is substantially free of its natural environment. As an example, an isolated protein, nucleic acid or VLP is substantially free of cellular material from the cell, tissue source or bacteria from which it was derived. The term also refers to preparations or components where the isolated biological molecule or component is sufficiently pure for pharmaceutical purposes; i.e., at least about 70% (w/w) pure, at least about 80% pure, at least about 90% pure, at least about 95% pure, at least about 96%, 97%, 98%, 99%, 99+% or 100% pure.

The “virus-like particle” (VLP) refers to a particle a particle comprising an interior core that is surrounded by a capsid comprising one or more coat proteins, where the particle is a non-infectious structural analogue of its parental virus. VLPs can be highly monodisperse, enabling their self-assembly into well-ordered 2D structures by, for example, simple evaporation-driven techniques. VLPs according to the present invention are non-replicative and non-infectious, lacking at least the gene(s) that encode the replication enzymes and typically also lacking the gene(s) responsible for viral attachment to or entry into the host cell. In certain embodiments, VLPs can include nanoparticles that have at least one dimension less than about 1000 nm. For example, VLPs can have at least one dimension ranging from about 10 to about 100 nanometers.

The present disclosure provides virus-like particles (VLPs) as well as methods of producing and using VLPs. Various embodiments provide systems and methods for using VLPs as targeted multifunctional nanocarriers for delivery of drugs, therapeutics, sensors, or imaging agents to various cell types. For example, by loading the VLP surface with one or more ligands that specifically bind to a target cell, VLPs can be used for targeted delivery of therapeutic or imaging agents, including for example, targeted delivery of therapeutic or imaging agents to cancer cells, such as hepatocellular carcinoma. VLPs can be rapidly produced in large quantities using in vivo or in vitro synthesis techniques.

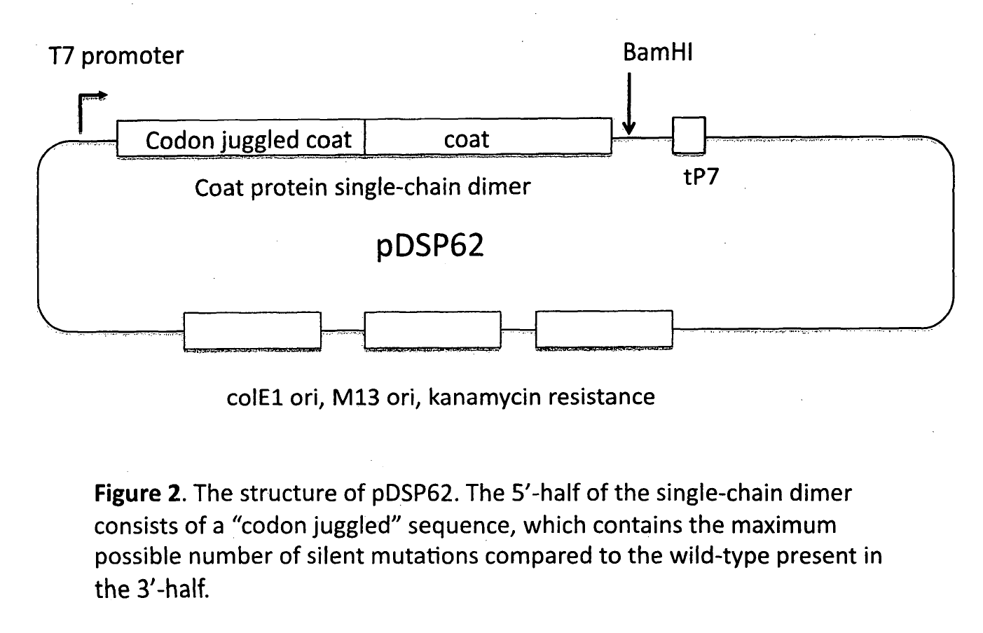

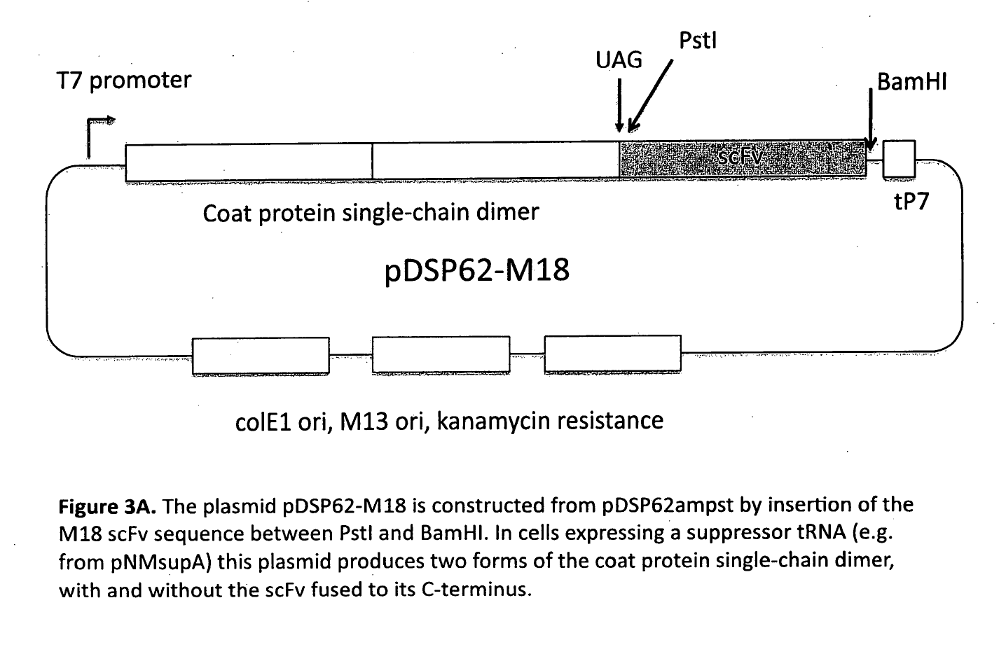

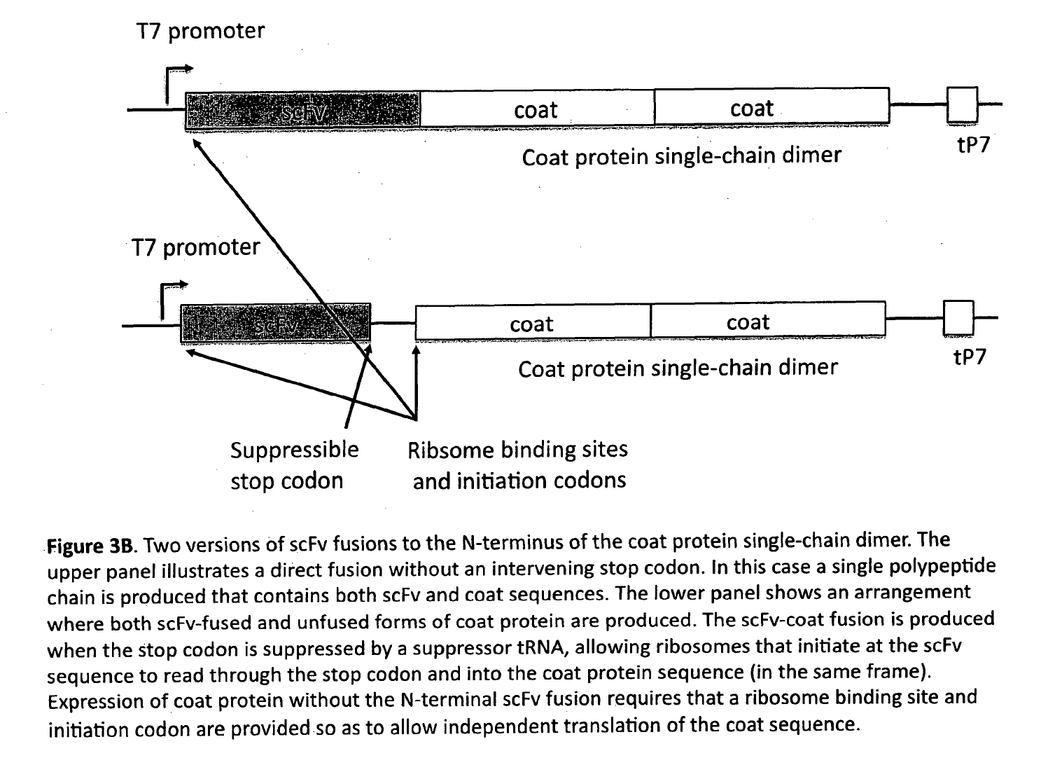

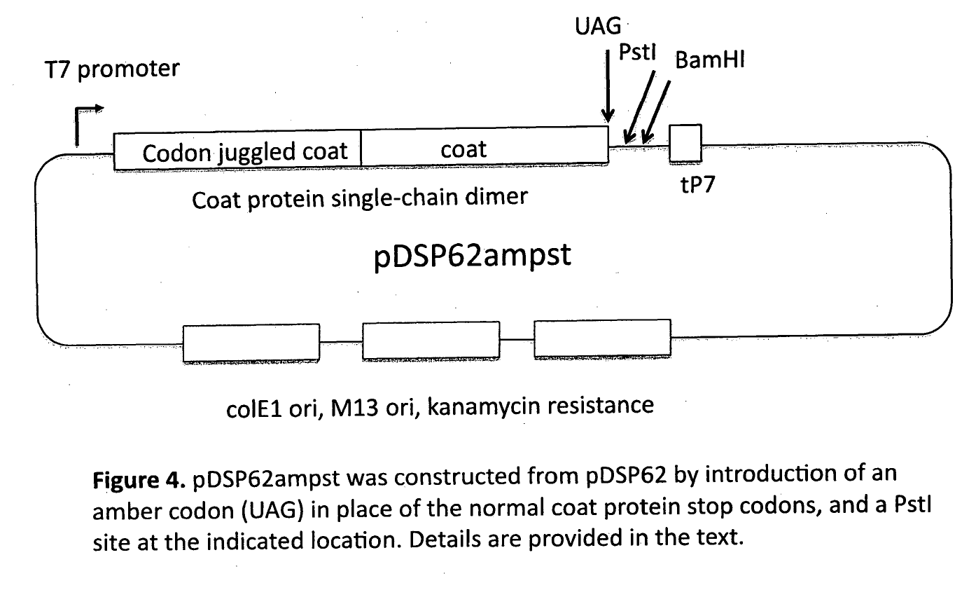

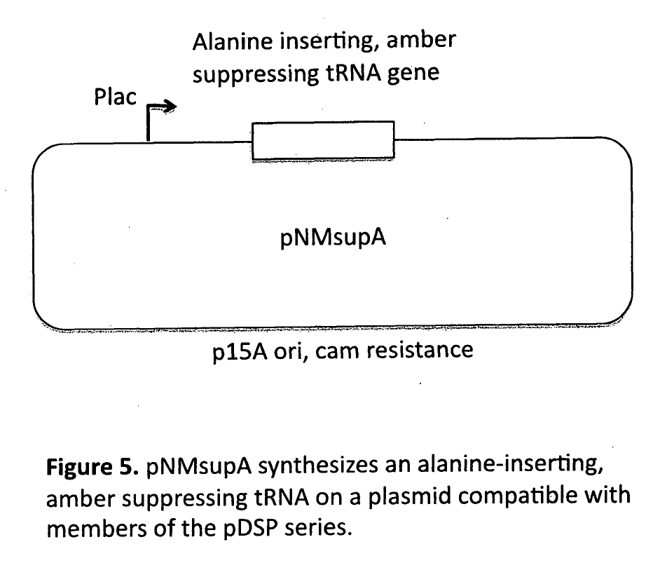

The term “single-chain dimer” refers to a normally dimeric protein whose two subunits have been genetically (chemically, through covalent bonds) fused into a single polypeptide chain. Specifically, in the present invention single-chain dimer versions of the MS2 coat protein was utilized. It is naturally a non-covalent dimer of identical polypeptide chains in which the N-terminus of one subunit lies in close physical proximity to the C-terminus of the companion subunit (see FIG. 2). Single-chain coat protein dimers were produced using recombinant DNA methods by duplicating the DNA coding sequence of the coat proteins and then fusing them to one another in tail to head fashion. The result is a single polypeptide chain in which the coat protein amino acid appears twice, with the C-terminus of the upstream copy covalently fused to the N-terminus of the downstream copy. Normally (wild-type) the two subunits are associated only through noncovalent interactions between the two chains. In the single-chain dimer these noncovalent interactions are maintained, but the two subunits have additionally been covalently tethered to one another. This greatly stabilizes the folded structure of the protein and confers to it a high tolerance of perturbations of its structure, including peptide insertions and N- and C-terminal fusions such as those as described above. Most embodiments according to the present invention make use of the single-chain dimer, but the wild-type (i.e. unduplicated) protein may also be employed.

The amino acid residues described herein are preferred to be in the “L” isomeric form. However, residues in the “D” isomeric form can be substituted for any L-amino acid residue, as long as the desired function is retained by the polypeptide. NH₂ refers to the free amino group present at the amino terminus of a polypeptide. COOH refers to the free carboxy group present at the carboxy terminus of a polypeptide.

The term “coding sequence” is defined herein as a portion of a nucleic acid sequence that directly specifies the amino acid sequence of its protein product. The boundaries of the coding sequence are generally determined by a ribosome binding (or Shine-Dalgarno) site and a translation initiation codon (usually AUG) in prokaryotes, or by the AUG start codon in eukaryotes located at the start of the open reading frame, usually near the 5′-end of the mRNA, and a translation terminator sequence (one of the nonsense codons: UAG, UGA, or UAA) located at and specifying the end of the open reading frame, usually near the 3′-end of the mRNA. A coding sequence can include, but is not limited to, DNA, cDNA, and recombinant nucleic acid sequences.

As briefly noted above, a “stop codon” or “termination codon” is a nucleotide triplet within messenger RNA that signals a termination of translation. Proteins are unique sequences of amino acids, and most codons in messenger RNA correspond to the addition of an amino acid to a growing protein chain—stop codons signal the termination of this process, releasing the amino acid chain. In the standard genetic code, there are three stop codons: UAG (in RNA)/TAG (in DNA) (also known as an “amber” stop codon), UAA/TAA (also known as an “ochre” stop codon), and UGA/TGA (also known as an “opal” or “umber” stop codon). Several variations to this predominant group are known. The use of a stop codon in the present invention will normally stop or terminate protein synthesis. However, there are mutations in tRNAs which allow them to recognize the stop codons, causing ribosomes to read through the stop codon, allowing synthesis of peptides encoded downstream of the stop codon [11-13]. For example, a mutation in the tRNA which recognizes the amber stop codon allows translation to “read through” the codon and produce full length protein, thereby recovering the normal form of the protein and “suppressing” the stop codon. Most often, suppression of stop codons is only partially efficient—often only a few percent of ribosomes are permitted to read though the stop codon. In some instances, however, suppression can be much more efficient. A few suppressor tRNAs simply possess higher intrinsic suppressions efficiencies. In other cases a weak suppressor can be made more efficient by simply expressing it at higher levels. In certain embodiments of the present invention, a stop codon is incorporated into transcriptional units in order to control the synthesis of peptides encoded within the transcriptional unit downstream of the stop codon. By providing for the controlled synthesis of tRNA which recognize the stop codon and allow synthesis of peptides downstream of the stop codon, coat protein may be produced which comprise a heterologous peptide within a population of coat proteins, the majority of which do not contain a heterologous peptide. The resulting VLPs which are assembled from this mixture of heterologous peptide containing wild-type (absence of heterologous peptide) coat proteins result in a much lower valency of heterologous presentation.

A “heterologous” region of a recombinant cell is an identifiable segment of nucleic acid within a larger nucleic acid molecule that is not found in association with the larger molecule in nature. A “heterologous” peptide is a peptide which is an identifiable segment of a polypeptide that is not found in association with the larger polypeptide in nature.

The present invention provides a method for adjusting the valency of foreign peptide and protein display. For some applications a low valency is preferred. In others high valency is desired. The valency of a VLP refers to the number of copies of a heterologous peptide or protein ligand displayed on the particles. A virus particle which exhibits “low valency” of a heterologous peptide, or in the present invention, at least an scFv, is a particle which displays an average of from fewer than one to up to about ten or more copies per VLP. VLPs which exhibit low valency are formed from a plurality of coat polypeptide dimers which are free of the scFv fusion and a minority of coat polypeptide dimers fused to the scFv, thus forming a mosaic VLP. Fusions of scFv's to either the N- or C-termini, especially the C-termini of coat protein are preferred, although insertions in the AB loop especially of single chain dimers may be accommodated in the present invention. Alternatively, low valency VLPs may be formed fro a mixture of wild-type coat polypeptide and dimer coat polypeptide which is fused to scFV peptide, thus forming a low valency VLP.

An “origin of replication”, used within context, normally refers to those DNA sequences that participate in DNA synthesis by specifying a DNA replication initiation region. In the presence of needed factors (DNA polymerases, and the like) an origin of replication causes DNA associated with it to be replicated. For example, a replication origin (used in a plasmid) endows many commonly used plasmid cloning vectors with the capacity to replicate independently of the bacterial chromosome. The presence on a plasmid of an additional origin of replication may confer the additional ability to replicate using that origin when bacterial cells such as E. coli cells are infected with a so-called helper phage, which provides necessary protein factors.

A “promoter sequence” is a DNA regulatory region capable of binding RNA polymerase in a cell and initiating transcription of a downstream (3′ direction) coding sequence. For purposes of defining the present invention, the promoter sequence includes the minimum number of bases or elements necessary to initiate transcription at levels detectable above background. Within the promoter will be found DNA sequences responsible for the binding of RNA polymerase and any of the associated factors necessary for transcription initiation. In bacteria promoters normally consist of −35 and −10 consensus sequences and a more or less specific transcription initiation site. Eukaryotic promoters will often, but not always, contain “TATA” boxes and “CAT” boxes. Bacterial expression vectors (usually plasmids or phages) typically utilize promoters derived from natural sources, including those derived from the E. coli Lactose, Arabinose, Tryptophan, and ProB operons, as well as others from bacteriophage sources. Examples include promoters from bacteriophages lambda, T7, T3 and SP6.

In bacteria, transcription normally terminates at specific transcription termination sequences, which typically are categorized as rho-dependent and rho-independent (or intrinsic) terminators, depending on whether they require the action of the bacterial rho-factor for their activity. These terminators specify the sites at which RNA polymerase is caused to stop its transcription activity, and thus they largely define the 3′-ends of the RNAs, although sometimes subsequent action of ribonucleases further trims the RNA.

An “antibiotic resistance gene” refers to a gene that encodes a protein that renders a bacterium resistant to a given antibiotic. For example, the kanamycin resistance gene directs the (synthesis of a phosphotransferase that modifies and inactivates the drug. The presence on plasmids (e.g. pDSP1, pDSP62, etc.) of a kanamycin resistance gene provides a mechanism to select for the presence of the plasmid within transformed bacteria. Similarly, the chloramphenicol resistance gene allows bacteria to grow in the presence of the drug by producing an acetyltransferase enzyme that inactivates the antibiotic through acetylation. In the present application chloramphenicol resistance is used to ensure the maintenance within bacteria of pNMsupA and M13CM1.

“Reverse transcription and PCR” are presented in as a means of amplifying the nucleic acid sequences of affinity-selected VLPs. “Reverse transcription” refers to the process by which a DNA copy of an RNA molecule (or cDNA) is produced by the action of the enzyme reverse transcriptase. In the present application, reverse transcription is used to produce a DNA copy of RNA sequences encapsidated with affinity-selected VLPs. The reverse transcriptase enzyme requires a primer be annealed to the RNA (see below).

The term “PCR” refers to the polymerase chain reaction, a technique used for the amplification of specific DNA sequences in vitro. The term “PCR primer” refers to DNA sequences (usually synthetic oligonucleotides) able to anneal to a target DNA, thus allowing a DNA polymerase (e.g. Taq DNA polymerase) to initiate DNA synthesis. Pairs of PCR primers are used in the polymerase chain reaction to initiate DNA synthesis on each of the two strands of a DNA and to thus amplify the DNA segment between two primers.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

As used herein, the term “coat protein(s)” refers to the protein(s) of a bacteriophage or a RNA-phage capable of being incorporated within the capsid assembly of the bacteriophage or the RNA-phage.

As used herein, a “coat polypeptide” as defined herein is a polypeptide fragment of the coat protein that possesses coat protein function and additionally encompasses the full length coat protein as well or single-chain variants thereof.

As defined hereinabove and as used herein, the term “virus-like particle of a bacteriophage” refers to a virus-like particle (VLP) resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host.

This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA bacteriophage coat protein: The capsid structure formed from the self-assembly of one or more subunits of RNA bacteriophage coat protein and optionally containing host RNA is referred to as a “VLP of RNA bacteriophage coat protein”. In a particular embodiment, the capsid structure is formed from the self assembly of 1-180 subunits.

A nucleic acid molecule is “operatively linked” to, “operably linked” or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

The term “stringent hybridization conditions” are known to those skilled in the art and can be found in Current Protocols in Molecular Biology, John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2.×SSC, 0.1% SDS at 50° C., preferably at 55° C., and more preferably at 60° C. or 65° C.

An “antibody single-chain variable fragment” (scFv) is a fusion of the variable regions of the heavy and light chains of immunoglobulins, linked together with a short linker, usually, but not exclusively, comprised of serines (S) or glycines (G). This chimeric molecule retains the specificity of the original immunoglobulin, despite removal of the constant regions and the introduction of a linker peptide. A scFv used in the invention may have a selective affinity to a wide variety of specific targets, including (but not limited to): pure recombinant proteins, a hapten, complex antigens such as viral coat proteins, toxins, environmental antigens, and cancer cell-related antigens. The V_{L} and V_{H} domains typically have lengths of about 110 amino acids (about 100-120 amino acids), and the linker is typically about 18-22 (15-25) amino acids. A variety of linker sequences have bee utilized but frequently they consist of a glycine/serine-rich sequence. One example is the linker of the anti-CD19 scFv (see SEQ ID No:1) and consists of the amino acid sequence: GGGGSGGGGSGGGGSGGGG. However, a variety of linker sequences are possible. For example, another, found in the anti-AF20 scFv (SEQ ID NO:2), is ADTTPKLEEGEFSEARV. The purpose of the linker is to covalently connect the N-terminus of one domain to the C-terminus of the other, and since the termini in the Fv are relatively far apart, a relatively long linker is needed. As long as it does not interfere with the folding, association, or function of the two domains, the sequence of the linker is irrelevant for most applications.

A “cancer cell-related antigen” can include (but is not limited to) an antigen implicated in neoplasm or neoplasia (i.e., is a “neoplastic cell”). As used herein, the term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991).

Many cell-surface proteins are expressed in a cell-specific manner. Some are differentially expressed on tumor cells, thus providing potential targets for VLP-scFv mediated drug and imaging agent delivery. Non-limiting examples of a “cancer cell-related antigen” therefore include CD99, carcinoembryonic antigen (CEA) (a membrane-bound glycoprotein expressed abundantly on epithelial cancerous cells), epidermal growth factor receptor (EGFR), VEGF, Ku86, HER2, the extra domain-B (EDB) of fibronectin, CD11c, tyrosine kinase (e.g. ErbB2), ICAM-1, MCAM/MUC18/CD146, CD3, CD19, cytotoxic T lymphocyte-associated antigen-4 (CTLA-4), the tumor stromal antigen fibroblast activation protein (FAP) and the extracellular domain of the 4-1BB ligand (4-1BBL). B-cell leukemias often express B-cell specific antigens like CD19 and CD22. Hepatocellular carcinomas generally overexpress a surface protein called AF20. Later in this application VLP-scFv's specific for CD19 and AF20 are described.

### Virus-Like Particles Pursuant to the Present Invention

The present invention is directed to virus-like phage particles as well as methods for producing these particles in vitro. The resulting VLPs can be used to conduct phage display in vitro. The invention makes it possible to increase laboratory complexity and reduce the time needed for iterative selection. The methods typically include producing virions in vitro and recovering the virions. As used herein, producing virions “in vitro” refers to producing virions outside of a cell, for instance, in a cell-free system, while producing virions “in vivo” refers to producing virions inside a cell, for instance, an Eschericia coli or Pseudomonas aeruginosa cell.

### Bacteriophages

The system envisioned here is based on the properties of single-strand RNA bacteriophages [RNA Bacteriophages, in The Bacteriophages. Calendar, R L, ed. Oxford University Press, 2005]. The known viruses of this group attack bacteria as diverse as E. coli, Pseudomonas and Acinetobacter. Each possesses a highly similar genome organization, replication strategy, and virion structure. In particular, the bacteriophages contain a single-stranded (+)-sense RNA genome, contain maturase, coat and replicase genes, and have small (<300 angstrom) icosahedral capsids. These include but are not limited to MS2, Qβ, R17, SP, PP7, GA, M11, MX1, f4, AP205, PRRI, Cb5, Cb12r, Cb23r, 7s and f2 RNA bacteriophages. The ssRNA bacteriophages have small genomes, typically about 3600-4200 nucleotides in length, encoding 4 proteins. In the MS2 family, the 4 proteins are a capsid coat protein, a replicase, a lysis protein and an attachment protein involved in the attachment of the phage to the host cell. In Qβ the 4 proteins are a capside coat protein, which also has lysis activity, a replicase, a minor varion protein, and an attachment protein.

For purposes of illustration, the genome of a particularly well-characterized member of the group, called MS2, comprises a single strand of (+)-sense RNA 3569 nucleotides long, encoding only four proteins, two of which are structural components of the virion. The viral particle is comprised of an icosahedral capsid made of 180 copies of coat protein and one molecule of maturase protein together with one molecule of the RNA genome. Coat protein is also a specific RNA binding protein. Assembly may possibly be initiated when coat protein associates with its specific recognition target an RNA hairpin near the 5′-end of the replicase cistron. The virus particle is then liberated into the medium when the cell bursts under the influence of the viral lysis protein. The formation of an infectious virus requires at least three components, namely coat protein, maturase and viral genome RNA, but experiments show that the information required for assembly of the icosahedral capsid shell is contained entirely within coat protein itself. For example, purified coat protein can form capsids in vitro in a process stimulated by the presence of RNA [Beckett et al., 1988, J. Mol Biol 204: 939-47]. Moreover, coat protein expressed in cells from a plasmid assembles into a virus-like particle in vivo [Peabody, D S., 1990, J Biol Chem 265: 5684-5689].

By way of example, MS2 VEPs self-assemble from 180 copies of a single coat protein (13.7 kDa) into a monodisperse, 27.5-nm capsid with icosahedral symmetry (T=3). The periodicity of the capsid, the presence of surface-accessible amino acids with reactive moieties (e.g. lysine and glutamic acid residues), and the tolerance of a genetically-fused coat protein dimer (the so-called single-chain dimer (25)) for at least 90% of pcptide insertions enables dense, repetitive display of peptides and antibody fragments via chemical conjugation. See, Carrico, et al., Chemical Communications: 1207-1207 (2008); Hooker, et al., Nano Letters 7(8):2207-2210 (2007); Kovacs, et al, Bioconjugate Chemistry, 18(4):1140-1147 (2007); Tong, et al, Journal of the American Chemical Society 131(31):11174-11178 (2009); Wu, et al., Bioconjugute Chemistry 6(5):587-595 (1995); Wu, et al., Nanomedicine: Nanotechnology, Biology and Medicine 1(1):67-76 (2005); and Wu, et al, Angewandte Chemie, 121(5):9657-9661 (2009).

Another attractive feature of MS2 VLPs is that their interior volume can be rapidly loaded with a variety of non-genomic materials using several approaches. Hollow capsids can be produced from native bacteriophages via base-catalyzed hydrolysis of the genome (Hooker, et al, Journal of the American Chemical Society, 126(12):3718-3719 (2004) or by expression of coat protein from a plasmid in transformed Escherichia coli. See, by expression of coat protein from a plasmid in transformed Escherichia coli (See, Tong, et al, Journal of the American Chemical Society 131(31):11174-11178 (2009); Pickett & Peabody, Nucl Acids Res., 21(19):4621-4626 (1993); and Rohrmann & Krueger, Biochemicand and Biophysical Research Communications, 38(3):406-413 (1970). The presence of 32 pores in the MS2 capsid, each of which is about 1.8-nm in diameter, enables diffusion of small molecules into the interior volume, a technique that has been employed to encapsidate antisense oligonucleotides, fluorescent molecules, chemotherapeutic drugs, and Gd-based contrast agents (See, Hooker, et al., Nano Letters 7(8):2207-2210 (2007); Tong, et al, Journal of the American Chemical Society 131(31):11174-11178 (2009); Wu, et al., Nanomedicine: Nanotechnology, Biology and Medicine 1(1):67-76 (2005); and Wu, et al, Angewandte Chemie, 121(5):9657-9661 (2009). 27, 29, 3 1-32).

### Coat Polypeptide

The invention described here is based on the virus-like particles produced when an RNA phage coat protein is synthesized from a plasmid. The coat polypeptide encoded by the coding region is typically at least 120, preferably, at least 125 amino acids in length, and no greater than 135 amino acids in length, preferably, no greater than 130 amino acids in length. It is expected that a coat polypeptide from essentially any single-stranded RNA bacteriophage can be used. Examples of coat polypeptides include but are not limited to the MS2 coat polypeptide, R17 coat polypeptide (see, for example, Genbank Accession No P03612), PRR1 coat polypeptide (see, for example, Genbank Accession No. ABH03627), fr phage coat polypeptide (see, for example, Genbank Accession No, NP_{—}039624), GA coat polypeptide (see, for example, Genbank Accession No. P07234), Qβ coat polypeptide (see, for example, Genbank Accession No. P03615), SP coat polypeptide (see, for example, Genbank Accession No P09673), f4 coat polypeptide (see, for example, Genbank accession no. M37979.1 and PP7 coat polypeptide (see, for example, Genbank Accession No P0363 0).

The VLPs comprise a protein shell, or capsid, comprised of coat protein monomers. In embodiments, the capsids of VLPs adopt icosahedrai structures. For example, icosahedral VLPs can be self-assembled from an integer multiple (T) of about 60 coat protein monomers into a hollow monodisperse protein nanoparticle. In other embodiments, the VLP capsid is formed from the self-assembly of 180 coat protein monomers. The VLP capsid can be modified in precise locations via genetic insertion or chemical conjugation, facilitating the multivalent display of targeting.

Typically, the coat protein of RNA bacteriophages assembles into a dimer of two identical subunits. It binds and encapsidates viral RNA, and also acts as translational repressor of viral replicase by binding to a RNA hairpin in the RNA genome. The coat protein from any bacteriophage can be used in the VLPs described herein. In certain embodiments, the bacteriophage coat protein is the coat protein from a single-stranded RNA bacteriophage, including but not limited to, MS2, Qβ, R17, SP, PP7, GA, M11, MX1, f4, AP205, PRRI, Cb5, Cb12r, Cb23r, 7s and f2, as otherwise described herein. The amino acid sequences of these bacteriophages are known. For example, the amino acid sequence of the MS2 coat protein corresponds to Accession No. NP_{—}040648.1, GI:9626313 and the amino acid sequence of the Qβ coat protein corresponds to the Accession No. P03615, GI:2507564. The MS2 coat protein is typically harvested via acetic acid or urea-driven disassembly of native MS2 bacteriophage (Wu, Bioconjugate Chemistry, supra).

The coat polypeptides useful in the present invention also include those having similarity with one or more of the coat polypeptide sequences disclosed above. The similarity is referred to as structural similarity. Structural similarity may be determined by aligning the residues of the two amino acid sequences (i.e., a candidate amino acid sequence and the amino acid sequence) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence can be isolated from a single stranded RNA virus, or can be produced using recombinant techniques, or chemically or enzymatically synthesized. Preferably, two amino acid sequences are compared using the BESTFIT algorithm in the GCG package (version 10.2, Madison Wis.), or the Blastp program of the BLAST 2 search algorithm, as described by Tatusova, et al. (FEMS Microbial Lett 1999, 174:247-250), and available at http://www.ncbi.nlm.nih.gov/blast/b12seq/b12.html. Preferably, the default values for all BLAST 2 search parameters are used, including matrix=BLOSUM62; open gap penalty=11, extension gap penalty=1, gap xdropoff=50, expect=10, wordsize=3, and optionally, filter on. In the comparison of two amino acid sequences using the BLAST search algorithm, structural similarity is referred to as “identities.” Preferably, a coat polypeptide also includes polypeptides with an amino acid sequence having at least 80% amino acid identity, at least 85% amino acid identity, at least 90% amino acid identity, at least 95% amino acid identity, at least 96% amino acid identity, at least 97% amino acid identity, at least 98% amino acid identity, at least 99% amino acid identity, at least 99+% amino acid identity to one or more of the amino acid sequences disclosed above. Preferably, a coat polypeptide is active. Whether a coat polypeptide is active can be determined by evaluating the ability of the polypeptide to form a capsid and package a single stranded RNA molecule. Such an evaluation can be done using an in vivo or in vitro system, and such methods are known in the art and routine. Alternatively, a polypeptide may be considered to be structurally similar if it has similar three dimensional structure as the recited coat polypeptide and/or functional activity.

The antibody single-chain variable fragment may be present at the amino-terminal end of a coat polypeptide, at the carboxy-terminal end of a coat polypeptide, or present elsewhere within the coat polypeptide. Preferably, the antibody single-chain variable fragment sequence is present at a location in the coat polypeptide such that the insert sequence is expressed on the outer surface of the capsid. In a particular embodiment, the antibody single-chain variable fragment sequence may be inserted into the AB loop regions the above-mentioned coat polypeptides. Examples of such locations include, for instance, insertion of the insert sequence into a coat polypeptide immediately following amino acids 11-17, or amino acids 13-17 of the coat polypeptide. In a most particular embodiment, the antibody single-chain variable fragment is inserted at a site corresponding to amino acids 11-17 or more particularly 13-17 of MS-2.

Alternatively, the antibody single-chain variable fragment may be inserted at the N-terminus or C-terminus of the coat polypeptide.

In order to determine a corresponding position in a structurally similar coat polypeptide, the amino acid sequence of this structurally similar coat polypeptide is aligned with the sequence of the named coat polypeptide as specified above.

In a particular embodiment, the coat polypeptide is a single-chain dimer containing an upstream and downstream subunit. Each subunit contains a functional coat polypeptide sequence. The antibody single-chain variable fragment may be inserted on the upstream and/or downstream subunit at the sites mentioned herein above, e.g., the A-B loop region of downstream subunit. In a particular embodiment, the coat polypeptide is a single chain dimer of an MS2 coat polypeptide.

The inner and/or outer surfaces of the protein capsid of the VLPs of the present invention which comprise scFv peptides also can be modified with one or more ligands, such as a targeting peptide, sugars, vitamins, nucleic acids (e.g., aptamers, siRNA, antisense oligonucleotides, etc.) and/or various cargos (e.g., drugs, including other cargoes as otherwise disclosed herein). For example, in addition to displaying scFV peptides, VLP capsids according to the present invention can be engineered to express or display non-native (or heterologous) peptides with known affinities. These peptides can, for example, (a) nucleate gold, zinc sulfide, etc. from precursor salt solutions (e.g., gold-binding peptide); (2) condense silica from silicic acid at neutral pH (e.g. R5 repeat units from silaffins); (3) detect the presence of surface antigens expressed by pathogens (e.g. LPS-reactive peptides); (4) produce monoclonal antibodies against a given pathogen and (5) target drug carriers to a specific cell type, including cells which are infected with bacteria, a virus (especially including HIV) or other infectious agent, or a cancer cell.

In one embodiment, the VLP expresses or displays a scFv peptide that has an affinity for a cancer cell, including solid tumors and leukemias. Preferably the peptide does not bind to non-cancerous cells or, alternatively, binds to the target cancer cell with at least a 1000-fold, at least 5,000-fold, at least 10,000-fold, or at least 20,000-fold higher affinity than for a non-cancerous control cell. The cancer cell includes but is not limited to liver, prostate, colorectal, breast, multiple myeloma, pancreatic, small cell carcinoma, acute myelogenous leukemia, chronic myelogenous leukemia, acute lymphoblastic leukemia, myelo-proliferative disease, nonsmall cell lung, small cell lung, chronic lymphoid leukemia, sarcoma, melanoma, lymphoma, thyroid, neuroendocrine, renal cell, gastric, gastrointestinal stromal, glioma, brain or brain, bladder or as otherwise described herein.

In other embodiments, the VLP expresses or displays a scFv peptide that has an affinity for a cell infected with a virus, including an adenovirus, an astrovirus, a hepadnavirus, a herpes virus, a papovavirus, a poxvirus, an areavirus, a bunyavirus, a calcivirus, a coronavirus, a filovirus, a flavivirus, an orthomyxovirus, a paramyxovirus, a picornavirus, a reovirus, a retrovirus, a rhabdovirus, or a togavirus, among others. In one preferred embodiment, the virus is a human deficiency virus, including HIV-1 or HIV-2. In preferred aspects of the invention, the scFv does not bind to uninfected cells or binds to a virally infected cell with at least 1000-fold, at least 5000-fold, at least 10,000-fold, or at least 20,000-fold or higher affinity than for an uninfected cell.

In certain embodiments according to the present invention, VLPs displaying scFv peptides with an affinity for a specific cell type are capable of selective binding, internalization, and cargo delivery to the target cell. In certain embodiments, a number of peptides can be fixed in repetitive patterns on the present scFv containing VLPs to provide multivalency as a means to further enhance binding to cells by the VLP. For example, MS2 VLPs can be modified with high densities (at least 30 ligands per VLP) of peptides, aptamers, and other low molecular weight (no more than 25 kDa) targeting ligands through chemical conjugation to surface-accessible amino acids with reactive moieties (e.g. lysine, glutamic acid, cystinie, aspartic acid, etc.) See, Carrico, et al., Chemical Communications, (2008), supra; Kovacs, Bioconjugate Chemistry, (2007), supra and Tong, et al, JACS, (2009), supra. Multivalent display of targeting ligands on a nanoparticle surface enhances the affinity of the monovalent ligand for the target cell or cellular receptor through collective binding effects that promote high avidity interactions between nanoparticles and the target cell surface (See, Tong, et al., JACS (2009), supra; Jiang, et al., Nat Nano 3(3): 145-1 50 (2008); Vance, et al., Advanced Drug Delivery Reviews 6 1(11):93 1-939 (2009); and Weissleder, et al., Nat Biotech 23(11): 141 8-1423 (2005).

Further, development of a complex VLP-based random peptide library can enable both identification of scFv and production of VLP drug carriers in a single, rapid, cost-effective process. In certain embodiments, the VLPs can be used as a platform for phage display, a combinatorial based approach that enables selection of scFv peptides from a complex library that bind to a specific material. The specific material can be organic, inorganic, or a biological material, including cells and/or cellular material.

For example, whole cancer cells, as well as receptors uniquely expressed or over-expressed by cancer cells can be used in the affinity selection process. On the other hand, noncancerous cells and/or tissue can be used in counter-selections, and various buffer conditions (e.g. ionic strength or addition of detergents) can be employed to increase the selection stringency.

The scFv heterologous protein may be attached to the coat protein at the N-terminus or C-terminal of the coat protein or elsewhere within the coat protein, such as within the A-B loop region, as discussed in U.S. Publication 200910054246, the disclosure of which is hereby incorporated by reference in its entirety. In certain embodiments, the scFv heterologous protein is attached such that it is expressed or displayed on the outer surface of the bacteriophage capsid. In other preferred embodiments, coat protein is a single-chain dimer as otherwise described herein comprising an upstream and a downstream unit. Each subunit contains a functional coat protein sequence. The heterologous peptide may be inserted into the upstream and/or downstream subunit at the sites mentioned above, e.g., and preferably, at the carboxy terminus of the downstream subunit. In preferred aspects of the invention, the coat protein is a single chain dimer of an MS2 or Qβ coat protein.

In certain embodiments, VLPs according to the present invention are used as targeted cargo delivery systems and can have external and internal (in many instances preferably, internal) modification of the capsids. For example, various chemistries can be used to modify the exterior and interior capsid surfaces with various organic and inorganic compounds. For purposes of the present discussion, these modifications are discussed hereinbelow with particular reference to MS2 and/or Qβ coat proteins, but the methods and discussion is generally applicable to the other coat proteins which may be used in the present invention.

External capside modification of VLPs with scFv peptides can be performed using a crosslinker to chemically conjugate a peptide, an anticancer agent or other agent to surface amino acids containing functional groups (lysine, glutamic acid, aspartic acid, etc.) which can accommodate a linker. Internal capside modification may be afforded as otherwise discussed in detail herein.

Production of VLPs Relevant to Therapeutic and/or Diagnostic Targeting

VLPs of bacteriophages, including RNA bacteriophages, such as MS2 and/or Qβ, can be readily produced by expression of coat protein from plasmids in bacteria, allowing over-expression and genetic manipulation of the VLP using recombinant DNA technology. In certain embodiments, VLPs can be assembled from 180 identical copies of coat protein, each of which is highly tolerant of a wide-variety of ScFv or other peptide insertions and can be genetically manipulated to display a scFv in a surface loop or at the C- or N-terminus, preferably the C-terminus. Each MS2 or Qβ VLP bearing a scFv peptide insert can selectively encapsidate mRNA that templates its synthesis, providing the genotype-phenotype linkage necessary for the affinity selection of scFv from complex random sequence libraries in a process analogous to phage display. Furthermore, MS2 or Qβ VLPs can be constructed entirely in vitro, enabling the development of highly complex libraries (e.g. more than 1011 scFvs) and making it possible to automate the library construction and affinity selector, processes.

In certain embodiments, the disclosed affinity selection process and the delivery system can also be applied to subject matters as described in a related patent application Ser. No. 11/895,198 (U.S. Publication 2009/0054246), and entitled “A Virus-Like Platform for Rapid Vaccine Discovery, which is incorporated by reference in its entirety herein. Vaccine Discovery,” which is hereby incorporated by reference in its entirety.

Bacteriophage VLPs, such as MS2 and/or Qβ bacteriophages, also self-assemble into complete capsids in the presence of nucleic acids and, thus, can be used to specifically encapsidate therapeutic RNA (e.g., siRNA, antisense oligonucleotides, microRNA, ribozymes, RNA decoys, aptamers), and other RNA-modified cargos, including one or more RNA-modified cytotoxic agents, including chemotherapeutic agents, one or more RNA modified imaging agents, (e.g. quantum dots). Typically, the nucleic acid is conjugated to the one or more cytotoxic agents or one or more imaging agents using a crosslinker molecule. It is within the ordinary skill in the art to select an appropriate crosslinker, many are commercially available and may be used according to the functionality (e.g, nucleophile and/or electrophile) of the compounds to be linked through the crosslinker. In one embodiment, the crosslinks molecule comprises a reactive group such as an NHS ester, that reacts with an amino group to form an amide bond. In another embodiment, the crosslinker molecule is a heterobifunctional crosslinker molecule that can react with sulfhydryl or amino groups, including, for example, AMAS, BMPS, GMBS, sulfo-GMBS, MBS, sulfo-MBS, SMCC, sulfo-SMCC, EMCS, sulfo-EMCS, SMPB, sulfo-SMPB, SMPH, LC-SMCC, Sulfo-KMUS, SM(PEG)n NHS-PEG-Maleimide Crosslinkers, SPDP, LC-SPDP, sulfo-LC-SPDP, SMPT, sulfo-LC-SMPT, SIA, SBAP, STAB, or sulfo-SIAB, among numerous others well known in the art. In certain embodiments, the crosslinking agent is cleavable via reduction or oxidation reactions which occur in a cell. Using crosslinkers that are cleavage via oxidation or reduction may assist in liberating cytotoxic agents in the cytosol of target cells, in particular, cancer cells. Exemplary cleavable crosslinkers, include, for example, SPDP, LC-SPDP, sclfo-LC-SPCP, SMPT, and sulfa-LC-SMFT, among others.

In certain aspects of the invention, an anticancer agents, such as a chemotherapeutic agent, e.g. doxorubicin, among numerous others may be conjugated to the pac site of MS2 using a heterobifunctional crosslinker molecule (e.g., NHS ester-maleimide Reagent or other crosslinking agent) to link a primary amine, alcohol, or carboxylic acid moiety present in the cytotoxic agent (e.g., an amine in the case of doxorubicin) to a nucleic acid molecule (RNA or DNA, including, for example, the pac site) that is modified with a 3′ or 5′ sulfhydryl group or other functional group that may be used to link the crosslinker molecule to the anticancer agent.

In exemplary embodiments, cargo components including, for example, drugs (as otherwise described herein, therapeutic RNA, quantum dots, gold nanoparticles, iron oxide nanoparticles, etc. can be linked to the thiolated or otherwise modified pac site and incorporated within the capsids of VLPs.

The efficacy and rate of capsid assembly are influenced and maximized in the presence of the MS2 translational operator, a 19-nucleotide RNA stem-loop (SEQ ID NO:8) that, via its interaction with coat protein, mediates exclusive encapsidation of the MS2 genome during bacteriophage replication (See, Wu, et al., Bioconjugate Chemistry (1995), supra; Pickett & Peabody, Nucl. Acids Res. (1993, supra); and Uhlenbeck, Nature Structural Biology (1998), supra). The MS2 operator, or pac site, can promote efficient encapsidation of non-genomic materials, such as the A-chain of ricin toxin, within the interior volume of MS2 VLPs upon conjugation of the pac site to the cargo of interest (Wu, et al., Bioconjugate Chemistry (1995), supra and Wu, et al., Nanomedicine (2005), Supra. MS2 VLPs will also, however, encapsidate RNA hairpins with sequences that differ from that of the native operator (Uhlenbeck, Nature Structural Biology, 1998, supra), as well as heterologous nucleic acids, including single- and double-stranded RNA and DNA less than 3 kbp in length. Accordingly, the sequence of the pac site can be modified as long as the modification does not prevent the RNA molecule from inducing VLP self assembly of the coat proteins. By way of further example, the pac site can further comprise a spacer molecule such as a trimeric to nonameric polyU nucleotide.

Using methods well-known in the art, a polyethylene glycol moiety may be attached to the VLPs described herein. PEGylation helps to minimize proteolytic degradation, reduce the humoral immune response against the capsid protein, and reduce non-specific interactions with non-target cells and help to increase the circulation half-life and enhance bioavailability of the encapsiated cargo (Kovacs, Bioconjugate Chemistry (2007), supra. Masking the surface of a nanoparticle, such as a VLP, typically interferes with ligand binding and reduces specific affinity for a target cell. The use of polyethylene glycol to modify VLPs does not substantially affect the specific affinity of scFV containing VLPs for their target cells. affinity of VLPs for their t˜rgect ells.

### Targeted Delivery

The VLP delivery system described herein allows the simultaneous delivery of single or multiple types of chemically disparate therapeutic and/or imaging agents to cells, especially including cancer cells, using VLPs, including RNA bacteriophages, such as MS2 or Qβ as otherwise described herein. The VLPs according to the present invention can be modified with high densities of scFv that has an affinity for a cell, including a cancer cell. ScFvs which are genetically inserted into the VLP coat protein (fusion protein) are displayed on the VLP surface which displays high specific surface binding to the cell. These VLPs are internalized by the targeted cells, but not non-targeted (normal) cells. MS2 VLPs, furthermore, naturally self-assemble in the presence of RNA, thus enabling specific encapsidation of therapeutic RNAs (which may be conjugated to anticancer agents, etc.), as well as any molecule or nanoparticle (less than 16-nm in diameter) that can be surface-modified with RNA. Specific internalization of targeted VLPs enables selective delivery of a variety of cytotoxic (e.g., chemotherapeutic drugs, siRNA cocktails, and protein toxins, among others) and imaging agents (e.g, quantum dots) to targeted cells without affecting the viability of normal cells and other control cells in vitvo. The present invention demonstrates that VLPs, and in particular MS2 VLPs possess a unique combination of features, which enables their use as a flexible, robust system for targeted delivery of therapeutic and imaging agents to cancer and other disease states and justifies their development as a nanocarrier for numerous therapeutic and diagnostic applications of general applicable. Thus, the present invention may be used to deliver diverse cargos to a variety of cell types.

The preferred MS2 coat protein conjugated to a high affinity targeting scFv peptide can bind to RNA and spontaneously encapsidate RNA-drug complexes for use in directing therapeutic agents to specific target cells, such as cancer cells. For example, MS2 capsids bearing scFvs for targeting peptides on the surface of cells can bind to the target cells with high affinity at various scFv peptide densities, while maintaining a low or negligible affinity for a control cell line. Other scFv targeting peptides can be used. In certain embodiments, multiple targeting scFv peptides can be incorporated into VLPs, and the use of multiple targeting scFv peptides can help mitigate an immune response. In certain embodiments, the high affinity of MS2 VLPs for a particular cell can be retained when two different targeting scFv peptides are used simultaneously. The affinity selection process can enable identification of numerous different targeting scFv peptides, which through PCR and.

Pursuant to the present invention, targeted VLPs can be rapidly endocytosed by the target cell and routed to lysosomes selectively (they are not internalized by human normal non-targeted cells. Therefore, targeted VLPs can be used as a biocompatible carrier to deliver therapeutic and/or imaging agents to targeted human cells without affecting the viability of non-targeted cells. Targeted VLPs, can have at least a 10,000-fold or 20,000-fold higher affinity for the targeted cell than for the non-targeted human. This high affinity can be facilitated by multivalent peptide display (further including a separate binding peptide into or onto the scFv containing VLP). Note that the affinity of MS2 VLP for certain cells may decrease as scFv peptide density decreases. However, a peptide concentration of about 10-60, about 20 to 50, about 30 to 40 scFV peptides per VLP can be sufficient to achieve maximum specificity.

In certain embodiments according to the present invention the scFv containing VLPs can be endocytosed by targeted cells, localized in endosomes and/or lysosomes with a certain period of time. In certain embodiments, the scFv containing VLP can be endocytosed or internalized with the targeted cell in a short period of time ranging from less than an hour to several hours.

The localization of VLPs within lysosomes favorably promotes disassembly of the protein capsid, thereby releasing the cargo components. For example, cargo components, including gold and iron oxide nanoparticles, quantum dots, anticancer agents, and siRNA that silences cyclin A expression, cholera toxin A chain, and ricin toxin A chain can be encapsidated within MS2 VLPs, i.e., chemically conjugated to a specific RNA sequence present in the bacteriophage genome that initiates the capsid assembly, and can then be released upon internalization within human hepatocarcinoma cells. In other embodiments, when using a scFv peptide that targets the VLP to lysosomes, the coat protein of the VLP further comprises a polypeptide that induces osmotic swelling and destabilizes the membrane of lysosomes at a pKa of 6. The presence of the polypeptide in the capsid of the VLP helps to prevent the degradation of the VLP cargo within the lysosome. In one embodiment, the polypeptide comprises the amino acid sequence of SEQ ID NO:6.

In certain embodiments according to the present invention, the in vitro release of cargo components to human cells can take a certain period of time. In certain preferred embodiments, targeting MS2 VLPs internalized by target cells can release their cargo into the cytosol within, for example, about 2-6 hours, about 4 hours.

In various embodiments according to the present invention, cytotoxicity and cancer therapy induced by the therapeutic VLP's according to the present invention. In the present invention, therapeutic VLPs having anticancer agents, including cytotoxic agents as cargo, may be used to effect very specific anticancer therapy in numerous cancers in patients.

VLPs according to the present invention, especially including VLPs based upon MS2 and Qβ bacteriophages, are capable of simultaneous encapsidating and delivering multiple therapeutic and diagnostic agents. In certain embodiments, VLPs according to the present invention comprise one or more anticancer agent, resulting in an extremely favorable LC₉₀ value. In other embodiments, VLPs according to the present invention are loaded with a cocktail of siRNA, which silences expression of various cyclins (cyclin A2, cyclin B1, cyclin D1 or cyclin E1) and administered to a patient. Such VLPs can induce apoptosis in targeted cells within a short period of several hours, without impacting the viability of non-targeted (normal) cells. Furthermore, the siRNA cocktail, when delivered using VLPs according to the present invention, induces selective growth arrest and apoptosis in targeted cells without affecting the viability of normal cells. This effect can be facilitated by modifying the siRNA with a nuclear localization sequence, such as the NLS derived from the M9 domain of the heterogenous nuclear ribonucleoprotein A1: NQSSNFGPMKGGNFGGRSSGPYGGGGQYFAKPRNQGGY (SEQ ID NO:7). The NLS directed the siRNA to the nucleus of the target cell.

In other embodiments the scFv containing VLPs of the present invention are loaded with a toxin, such as the A chain of the ricin toxin to target cells to be eliminated. In yet another embodiment, VLPs according to the present invention may further comprise a polypeptide (SEQ ID NO: 6) that induces osmetic swelling and destabilizes the membrane of lysosomes at a pKa of 6. The presence of the polypeptide in the capsid of the VLP helps to prevent the degradation of the ricin A-chain once it is endocytosed by the target cell and transported to lysosomes within the target cell.

In another embodiment, the targeting VLPs can be used to treat any type of cancer. scFvs specific for prostate specific membrane antigen (PSMA) may be used to target chemotherapeutic agents to the prostate of a patient or to deliver siRNA that silences expression of the androgen receptor resulting in apoptosis of prostate cancer cells. scFvs specific for antigens expressed by various cancer cells may be used to target therapeutic compounds and/or diagnostic compounds to the cancer tissue, in vitro and in vivo.

### Pharmaceutical Compositions

The VLPs according to the present invention are suitable for pharmaceutical use and administration to patients. A typical pharmaceutical composition comprises a VLP, as otherwise described herein, in combination with at least pharmaceutically acceptable carrier, additive or excipient.

A pharmaceutical composition is formulated to be compatible with its intended route of administration. Methods to accomplish the administration are known to those of ordinary skill in the art. Examples of administration of a pharmaceutical composition according to the present invention include, for example, oral ingestion, inhalation, intravenous, intraperitoneal, intramuscular, intracavity, subcutaneous, cutaneous, or transdermal.

Solutions or suspensions used for cutaneous or subcutaneous application typically include at least one of the following components: a sterile diluent such as water, saline solution, fixed oils, polyethylene glycol, glycerine, propylene glycol, or other synthetic solvents; antibacterial agents, such as benzyl alcohol or methyl parabens; antioxidants, such as ascorbic acid or sodium bisulfite; chelating agents, such as ethylenediaminetetraacetic acid (EDTA); buffers such as acetate, citrate, or phosphate; and tonicity agents, such as sodium chloride or dextrose. The pH of the composition can be adjusted readily with pharmaceutically acceptable acids or bases. Such preparations may be enclosed in ampoules, disposable syringes, or multiple dose vials.

Solutions or suspensions used for intravenous administration include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must be stable under the conditions of manufacture and storage. Microorganism growth can be prevented using antibacterial and antifungal agents, e.g., parabens, chlorobutanol, phenol, ascorbic acid, etc, In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin.

Oral compositions include an inert diluent or edible carrier. The composition can be enclosed in gelatin or compressed into tablets. For the purpose of oral administration, the VLPs can be incorporated with excipients and placed in tablets, troches, or capsules. Pharmaceutically compatible binding agents or adjuvant materials can be included in the composition. The tablets, troches; and capsules, may contain (1) a binder such as microcrystalline cellulose, gum tragacanth or gelatin; (2) an excipient such as starch or lactose, (3) a disintegrating agent such as alginic acid, Primogel, or corn starch; (4) a lubricant such as magnesium stearate; (5) a glidant such as colloidal silicon dioxide; or (6) a sweetening agent or a flavoring agent.

The pharmaceutical composition according to the invention may be administered by a transmucosal or transdermal route. Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the VLPs can be delivered in an aerosol spray from a pressured container or dispenser, which contains a propellant (e.g., liquid or gas) or a nebulizer.

The VLPs according to the present invention are administered in therapeutically-effective amounts as described. Therapeutically effective amounts vary as a function of the disease to be treated, the subject's age, condition, sex, size and severity of medical condition. Appropriate dosage may be determined by a physician based on clinical indications. The VLP-containing composition may be given as a bolus dose to maximize the circulating levels of the VLPs for the greatest length of time. Continuous infusion may also be used after the bolus dose.

Non-limiting examples of dosage ranges that can be administered to a subject can be chosen from: 1 μg/kg to 20 mg/kg, 1 μg/kg to 10 mg/kg, 1 μg/kg to 1 mg/kg, 10 μgkg to 1 mg/kg, 10 μg/kg to 100 μg/kg, 100 μg/kg to 1 mg/kg, 250 μgkg to 2 mg/kg, 250 μgkg to 1 mg/kg, 500 μg/kg to 2 mg/kg, 500 μg/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 20 mg/kg to 25 mg/kg, 15 mg/kg to 25 mg/kg, 20 mg/kg to 25 mg/kg, and 20 mg/kg to 30 mg/kg (or higher). These dosages may be administered daily, weekly, biweekly, monthly, or less frequently, for example, biannually, depending on dosage, method of administration, disorder or symptom(s) to be treated, and individual subject/patient characteristics. Dosages can also be administered via continuous infusion (e.g., through a pump or other medical device). The administered dose depends upon depend on the route of administration and the pharmacokinetics of the VLPs as well as the agents which are incorporated with the VLP. For example, subcutaneous administration may require a higher dosage than intravenous administration.

In certain instances, it may prove to be advantageous to formulate compositions in dosage unit form for ease of administration and uniformity of dosage. Dosage unit form as refers to physically discrete units of pharmaceutical composition suited for the patient to be administered at a point in time. Each dosage unit contains a predetermined quantity of VLP calculated to produce a therapeutic effect in association with the carrier, additive or excipient. The dosage unit depends on the characteristics of the VLPs and the particular therapeutic effect to be achieved.

Toxicity and therapeutic efficacy of the composition can be determined readily by standard pharmaceutical procedures in cell cultures or experimental animals. e.g. by determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀.

Data obtained from cell culture assays and animal studies can be used to formulate a dosage range in humans. The dosage may vary within this range depending upon the composition used and the route of administration. For any VLP used in the methods described herein, the therapeutically effective dose can be estimated initially using cell culture assays. Animal models can be used to determine circulating plasma concentrations and IC₅₀ values (i.e., the concentration of VLPs that achieves a half-maximal inhibition of symptoms). The effects of any particular dosage can be monitored by a suitable bioassay.

### Preparation of Transcription Unit

The transcription unit of the present invention comprises an expression regulatory region, (e.g., a promoter), a sequence encoding a coat polypeptide and transcription terminator. The RNA polynucleotide may optionally include a coat recognition site (also referred to a “packaging signal”, “translational operator sequence”, “coat recognition site”). Alternatively, the transcription unit may be free of the translational operator sequence. The promoter, coding region, transcription terminator, and, when present, the coat recognition site, are generally operably linked. “Operably linked” or “operably associated with” refer to a juxtaposition wherein the components so described are in a relationship permitting them to function in their intended manner. A regulatory sequence is “operably linked” to, or “operably associated with”, a coding region when it is joined in such a way that expression of the coding region is achieved under conditions compatible with the regulatory sequence. The coat recognition site, when present, may be at any location within the RNA polynucleotide provided it functions in the intended manner.

The invention is not limited by the use of any particular promoter, and a wide variety of promoters are known. The promoter used in the invention can be a constitutive or an inducible promoter. Preferred promoters are able to drive high levels of RNA encoded by me coding region encoding the coat polypeptide Examples of such promoters are known in the art and include, for instance, T7, T3, and SP6 promoters.

The nucleotide sequences of the coding regions encoding coat polypeptides described herein are readily determined. These classes of nucleotide sequences are large but finite, and the nucleotide sequence of each member of the class can be readily determined by one skilled in the art by reference to the standard genetic code.

Furthermore, the coding sequence of an RNA bacteriophage single chain coat polypeptide comprises a site for insertion of a heterologous peptide as well as a coding sequence for the heterologous peptide itself. In a particular embodiment, the site for insertion of the heterologous peptide is a restriction enzyme site.

In a particular embodiment, the coding region encodes a single-chain dimer of the coat polypeptide. In a most particular embodiment, the coding region encodes a modified single chain coat polypeptide dimer, where the modification comprises an insertion of a coding sequence at least four amino acids at the insertion site. The transcription unit may contain a bacterial promoter, such as a lac promoter or it ma contain a bacteriophage promoter, such as a T7 promoter and optionally a T7 transcription terminator.

In addition to containing a promoter and a coding region encoding a fusion polypeptide, the RNA polynucleotide typically includes a transcription terminator, and optionally, a coat recognition site. A coat recognition site is a nucleotide sequence that forms a hairpin when present as RNA. This is also referred to in the art as a translational operator, a packaging signal, and an RNA binding site. Without intending to be limiting, this structure is believed to act as the binding site recognized by the translational repressor (e.g., the coat polypeptide), and initiate RNA packaging. The nucleotide sequences of coat recognition sites are known in the art. Other coat recognition sequences have been characterized in the single stranded RNA bacteriophages R17, GA, Qβ, SP, and PP7, and are readily available to the skilled person. Essentially any transcriptional terminator can be used in the RNA polynucleotide, provided it functions with the promoter. Transcriptional terminators are known to the skilled person, readily available, and routinely used.

### Synthesis

VLPs are usually synthesized by expression of coat protein from a plasmid in bacteria, but as will be described in further detail below, the VLPs of the present invention may also be synthesized in vitro in a coupled cell-free transcription/translation system.

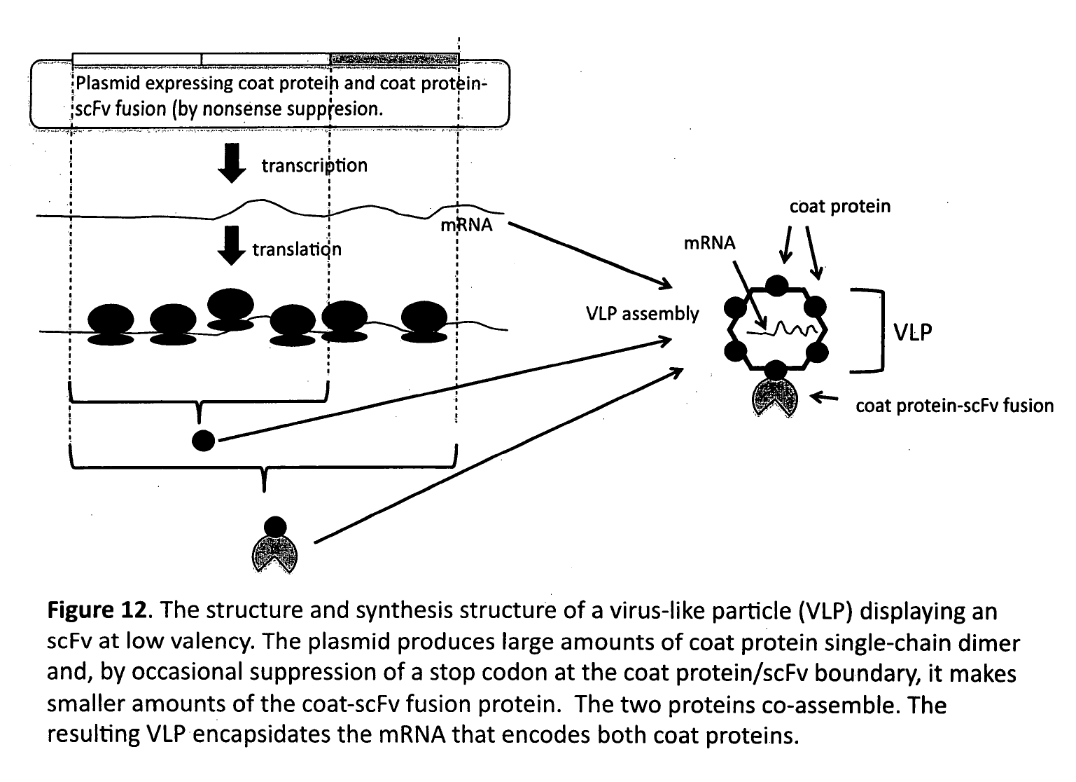

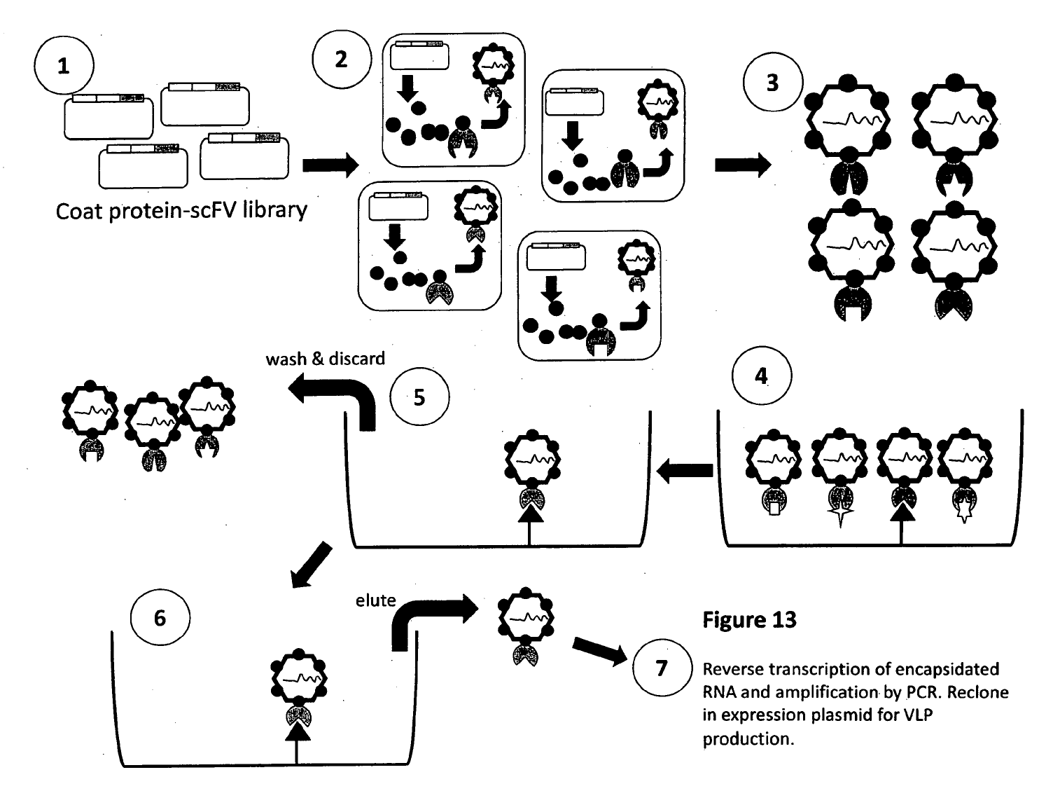

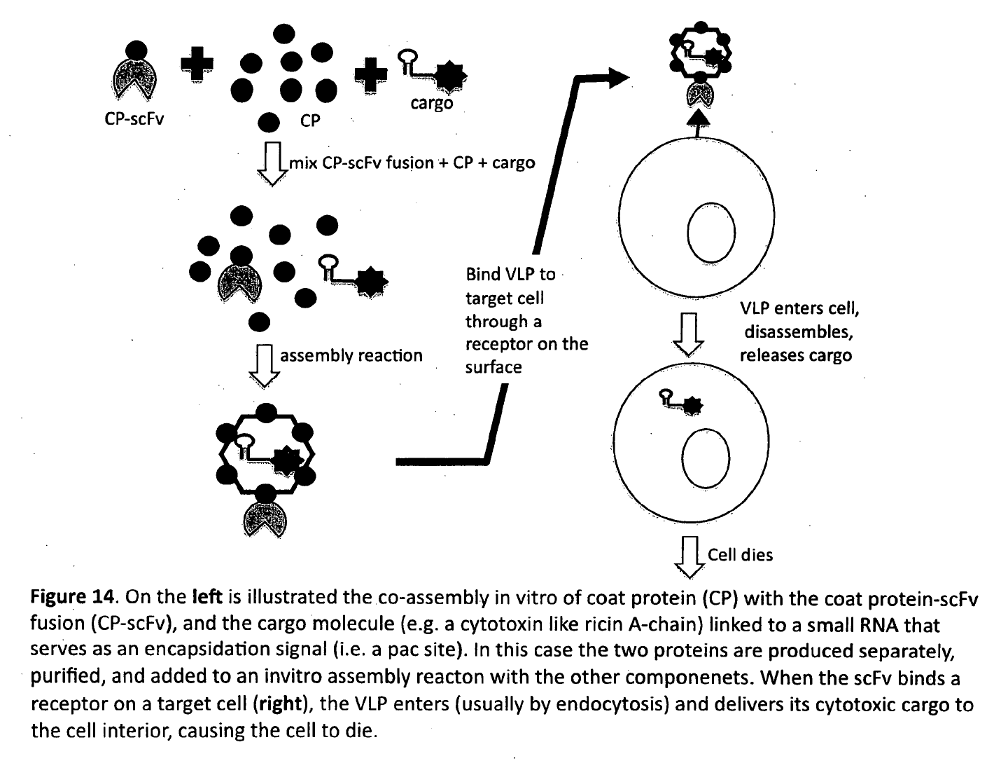

Approaches to synthesis of VLPs for various applications.
1. Synthesis of VLPs for affinity selection. For this application VLPs will normally be synthesized, whether in bacteria or by coupled transcription translation in vitro, as mosaics that assemble from coat protein and coat-scFv fusion according to the nonsense suppression scheme already described. Affinity selection depends on the recovery of selected sequences by reverse transcription and PCR amplification. This requires that both proteins be expressed from a single mRNA, which is then packaged into the resulting VLP. This is illustrated in FIGS. 12 and 13
2. Synthesis of VLPs for targeted delivery. These applications will normally require encapsidation of a molecular cargo (e.g. a cytotoxin or imaging agent), and in most cases particle loading will be accomplished by disassembly reassembly reactions. This means that the protein components of the VLP, namely coat protein and the coat-scFv fusion protein, may be prepared separately and then combined in desired ratios in the in vitro assembly reaction. This is illustrated in FIG. 14.

### Assembly of VLPs Encapsidating Heterologous Substances

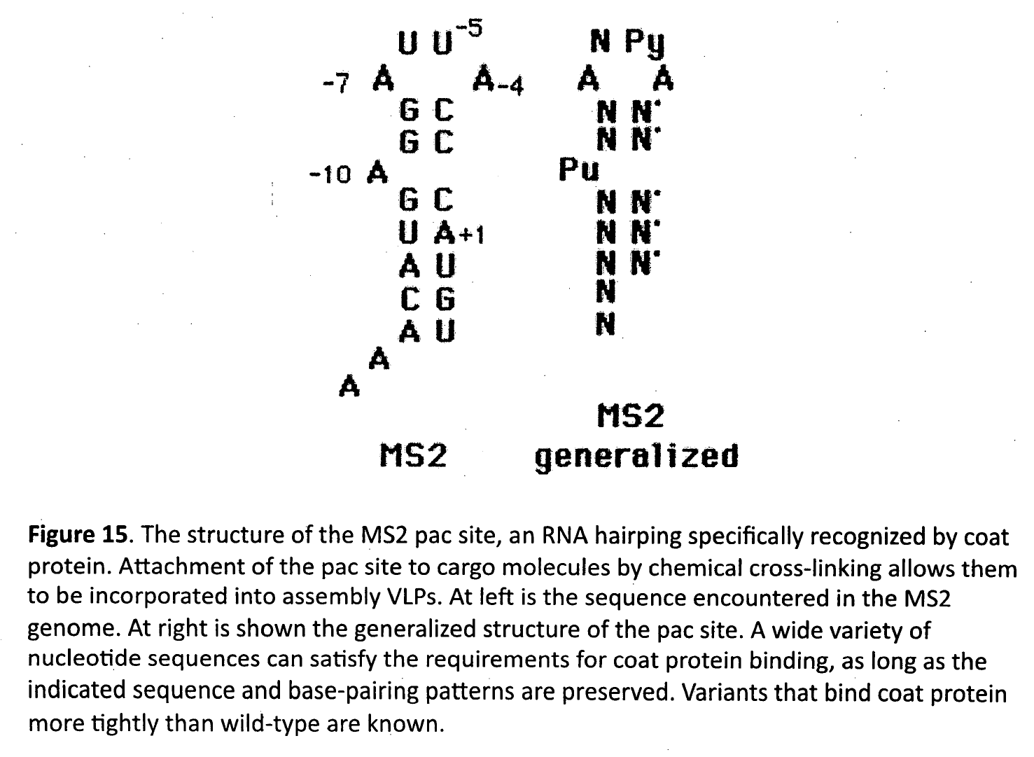

The decoration of a VLP's outer surface with a targeting scFv causes it to specifically bind and enter a targeted cell, but to be effective as a delivery vehicle for drugs, imaging agents, etc. it should be loaded with an appropriate molecular cargo. Non-limiting examples include cytotoxins like doxorubicin, ricin A-chain, and small interfering RNAs (i.e. siRNAs) that target the expression of proteins necessary for cell division (e.g. cyclins). RNA phage VLPs naturally encapsidate RNA, so the loading of particles with RNA cargos is simple matter of performing an in vitro assembly reaction in the presence of RNA. A variety of other cargo molecules, including, but not limited to proteins like ricin A-chain, small molecule drugs like doxorubicin, and imaging agents like quantum dots are typically modified by chemical attachment of an RNA molecule, usually a packaging signal called a pac site (see FIG. 15) which facilitates their uptake into the assembling VLP in vitro.

In vitro assembly reactions are conducted using purified VLPs or virus particles, which have been disaggregated by denaturation, using for example acetic acid or urea. When diluted in the presence of RNA or an RNA-linked cargo into solutions that favor protein refolding and VLP Assembly (e.g. neutral pH, reduced denaturant concentration) the cargo molecules are incorporated into the hollow interior of the VLP. A preferred RNA for these assembly reactions is the so-called “translational operator” or “pac” site, a small (e.g. about 20 nucleotide) RNA sequence which is tightly bound by coat protein. Such RNAs have been characterized for a number of RNA phages and are thought to mediate efficient assembly and encapsidation. It should be noted that many different RNA sequences can serve the function of the pac site. Those that bind coat protein most tightly are variations on the structure illustrated in FIG. 15.

The VLP has pores at the 5-fold and quae-6-fold symmetry axes that permit the entry of some cargo molecules into the intact particle without the need for disassembly-reassembly. Some small molecule drugs (e.g. doxorubicin) when conjugated to a small RNA (e.g. MS2 translational operator RNA) may diffuse directly into the VLP where it will remain due to specific interaction with coat protein on the VLP interior surface.

### Targeted Drug Delivery and Biomedical Imaging

To facilitate targeted drug delivery, the present invention provides a vehicle which comprises one or more pharmaceutically-active agents (e.g. a pharmaceutically active polypeptide, protein, small molecule, gene sequence, or vaccine) and a VLP as described herein. The invention may be used for cell-type specific delivery of drugs by employing VLPs which will deliver compounds to specific cells. Thus, the invention further includes therapeutic methods employing VLPs to deliver compounds (e.g., drugs) to specific cell-types in an organism. The particles may also be loaded with or otherwise hound to image contrast enhancing agents, which may bind specific cell or tissue types, permitting them to be imaged by a variety of methods.

Examples of pharmaceutically-active agents which may be used in conjunction with the invention include, but are not limited to, nucleoside analogues (e.g., acyclovir, gancyclovir, idoxuridine, ribavirin, vidaribine, zidovudine, didanosine and 2′,3′-dideoxycytidine (ddC), amantadine, etc.), antibiotics (e.g., sulphonamides, such assulanilamide, sulphacarbamide and sulphamethoxydiazine; penicillins, such as 6-aminopenicillanic acid, penicillin G and penicillin V; isoxazoylpenicillins, such as oxacillin, cloxacillin, flucloxacillin; α-substituted benzylpenicillins, such as ampicillin, carbenicillin, pivampicillin and amoxicillin; acylaminopenicillins, such as mezlocillin, azlocillin, piperacillin and apalicillin; tetracyclines, such as tetracycline, chlortetracycline, oxytetracycline, demeclocycline, rolitetracycline, doxycycline and minocycline; chloramphenicols, such as chloramphenicol and thiamphenicol; gyrase inhibitors, such as nalixidic acid, pipemidic acid, norfloxacin, ofloxacin, ciprofloxacin and enoxacin; tuberculosis agents, such as isoniazid; cytokines, such as interleukin 2, interferon α-2a, interferon α-2b, interferon β-1a, interferon β-1b, and interferon γ-1b, etc.

Further non-limiting examples of pharmaceutically-active agents which include chemotherapeutic agents is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an MFR-TK inhibitor, an anti-IMF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovornab, Lep-etu, nolatrexed, azd2171, batahulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951 aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat. COL-3, neovastat, BMS-275291 squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

Further non-limiting examples of pharmaceutically-active agents which include anti-viral agents, in preferred aspects, anti-HIV agents is one or more members selected from the group consisting of nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20 (pentafuside) and Fuzeon (enfuvirtide), among others, and mixtures thereof.

In one embodiment, the MS2 VLP is a hollow sphere with an internal diameter on the order of about 1 to about 100 nm, about 5 to about 50 nm, about 10 to about 25 nm, about 20 nm. In a particular embodiment, the VLP also comprises a drug, e.g., a protein toxin to be delivered and optionally a ligand that binds to cell-type specific receptors. The internal composition of such a particle may be controlled by specifically loading it, for example, with a protein toxin like ricin, by coupling it to a synthetic translational operator mimic. By conferring the ability to bind cell type-specific receptors to the outer surface of such particles, it is possible to target delivery of the toxin (or other drug) to selected cell types. In a related aspect, the VLP comprising the coat polypeptide dimer may actually encapsidate a heterologous substance such as a bacterial toxin, adjuvant or immunostimulatory nucleic acid.

### Biomedical Imaging Agents

In the same way that drugs can be targeted to specific cell types, so could contrast agents for magnetic resonance imaging be delivered to specific cells or tissues, potentially increasing enormously the diagnostic power of MRI. In fact, MS2 particles have already been labeled with gadolinium to greatly increase MRI contrast [Anderson et al., 2006, Nano Letters 6(6), 1160-1164]. Thus, in a particular embodiment, such particles could be targeted to specific sites by displaying appropriate receptor-specific peptides on their surfaces. In a related aspect, the VLP comprising the coat polypeptide dimer may actually encapsidate the imaging agent. Alternatively, the VLP may be modified to link the CLP to an imaging agent through a linker.

### VLP Populations

The libraries which are constructed pursuant to the present invention are antibody libraries. The individual members of the library each display a different scFv, with a different amino acid sequence in its binding site, and hence a different binding specificity. Such libraries can be constructed, inter alia, as cDNAs of antibody-encoding mRNAs from immune system cells (e.g. B-cells), or by randomizing by site-directed mutagenesis techniques the amino acid sequence of the antibody combining site of a single scFv.

### Uses of VLPs and VLP Populations

There are a number of possible uses for the VLPs and VLP populations of the present invention. As will be described in further detail below, specific scFv-VLPs may be used as, therapeutic agents, alone or in combination with bioactive agents, drug delivery devices, biomedical imaging agents and self-assembling nanodevices, among numerous others. It is also anticipated that the ability to display scFv's on RNA phage VLPs will facilitate the production of complex scFv libraries from which antibodies with desired activities may be isolated by affinity-selection.

Antibody Libraries on RNA Phage VLPs.

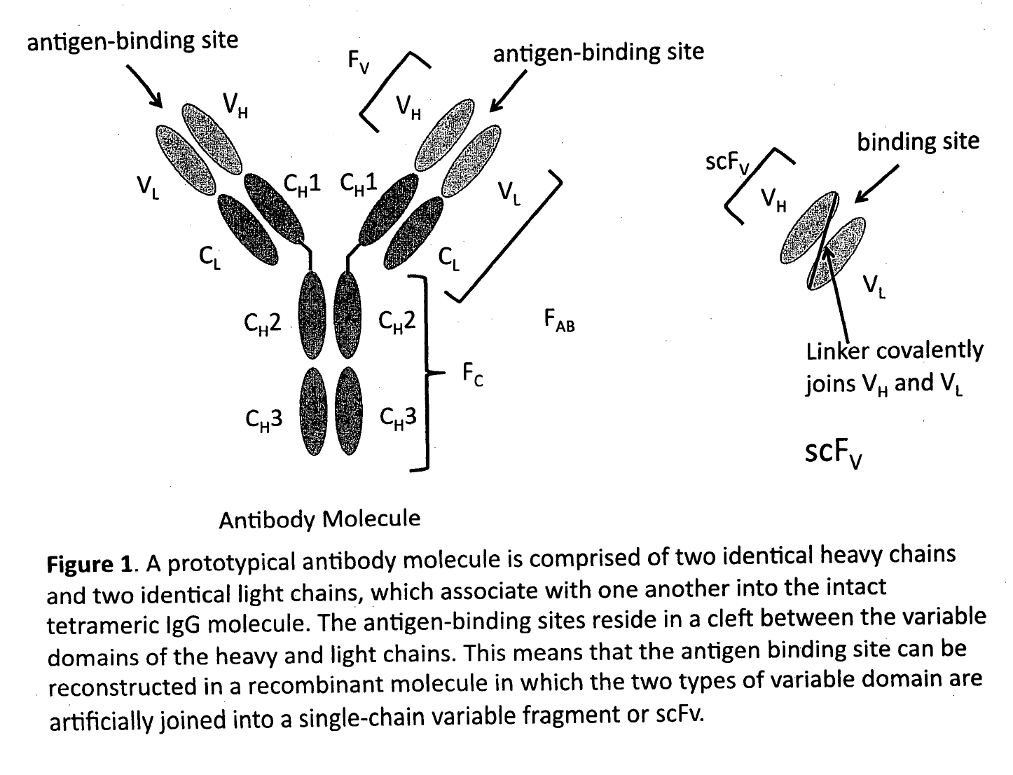

Diverse antibodies have a similar domain structure illustrated in FIG. 1. The sites where antigen is bound reside at the two tips of the Y-shaped molecule. Antibody molecules recognize diverse antigens by varying the amino acid sequences within their antigen-binding sites. The structural framework of different antibodies is relatively conserved, but the amino acids present within the antigen binding sties naturally vary enormously. Methods exist in the art for the production of scFv libraries using several different methods. For some examples, references xx-xx may be consulted. The general idea is to create a complex mixture of scFvs, either by taking advantage of existing diversity in the natural antibody repertoire, or to introduce new diversity in a pre-existing scFv framework by mutation of the so-called complementarity determining regions (CDRs), which determine the structure and ligand binding specificity of a given antibody binding site.

Mao, S, Gao, Changshou, Lo, C-H, Wirsching, P, Wong, C-H, and Janda, K D. (1999) Phage-display library selection of high-affinity human single-chain antibodies to tumor-associated carbohydrate antigens sialyl Lewis^{x} and Lewis^{x}. Proc. Natl. Acad. Sci. USA 96:6953-6958.
Soderling, E, Strandberg, L, Jirholt, P, Kobayashi, N, Alexeiva, V, Aberg, A-M, Nilsson, A, Jansson, B, Ohlin, M, Wingren, C, Danielsson, L, Carlsson, R, Borrecaeck, C A K (2000) Recombining germline-derived CDR sequences for creating diverse single-framework antibody libraries. Nature Biotechnology 18:852-856.
Tanaka, T, Chung, G T Y, Forster, A, Lobato, M N and Rabbitts, T H. (2003) De novo production of diverse intracellular antibody libraries. Nucleic Acids Res. 31:e23.

Conventional phage display methods (e.g. using filamentous phages) present scFv's as fusions to a viral structural protein (typically pIII). Libraries of diverse scFv sequences have been constructed and subjected to affinity selection (e.g. using biopanning) as a means of identifying scFv's with desired binding activities. However, using such an approach it is rather cumbersome to construct high sequence complexity scFv libraries in filamentous phage. The present invention provides a means to overcome such a large barrier to higher library complexity by eliminating the need for transformation of bacteria. Existing phage display systems rely on replication in bacteria (usually E. coli). Libraries are constructed in vitro by recombinant DNA techniques (e.g. by ligation of scFv sequences to phagemid DNA) and then introduced into E. coli where they replicate and produce recombinant viruses. Replication in E. coli is in fact necessary due to the complexities of the phage life cycle. Of course recombinant MS2 VLPs can be constructed by analogous methods, where libraries are produced by recombinant DNA, then cloned in bacteria where the VLPs are synthesized. Because of their simple composition, however (they consist of a single coat protein and the RNA that encodes it), the VLPs of RNA phages can alternatively be produced entirely in vitro, without the need for transformation of bacteria, thus significantly simplifying the approach. This means that the DNA templates for VLP synthesis can be constructed by in vitro methods involving restriction endonucleases and DNA ligases (as in standard recombinant DNA methods), or by assembly PCR, or by some combination of these methods without the need to introduce the DNA into bacteria for replication. Instead the resulting DNA templates can be directly employed in a coupled transcription/translation reaction where VLPs are synthesized in vitro. To ensure that each individual VLP-scFv species encapsidates its mRNA the templates are encapsulated in the aqueous microdroplets of a water oil emulsion before initiating the VLP synthesis reaction, thus ensuring the soupling of phenotype and genotype that is essential to the affinity selection method.

### Synthesis

In a particular embodiment, the populations of the present invention may be synthesized in a coupled in vitro transcription/translation system using procedures known in the art (see, for example, U.S. Pat. No. 7,008,651 Kramer et al., 1999, Cell-free coupled transcription-translation systems from E. coli, In. “Protein Expression. A Practical Approach”, Higgins and Hames (eds.), Oxford University Press). In a particular embodiment, bacteriophage T7 (or a related) RNA polymerase is used to direct the high-level transcription of genes cloned under control of a T7 promoter in systems optimized to efficiently translate the large amounts of RNA thus produced [for examples, see Kim et al., 1996, Eur J Biochem 239: 88 1-886; Jewett et al., 2004, Biotech and Bioeng 86: 19-26].

It is possible in a mixture of templates, particularly in the population of the present invention, different individual coat polypeptides, distinguished by their fusion to different peptides, could presumably package each other's mRNAs, thus destroying the genotype/phenotype linkage needed for effective phage display. Moreover, because each capsid is assembled from multiple subunits, formation of hybrid capsids may occur. Thus, in one preferred embodiment, when preparing the populations or libraries of the present invention, one or more cycles of the transcription/translation reactions should be performed in water/oil emulsions [Tawfik et al., 1998, Nat Biotechnol 16: 652-6]. In this now well-established method, individual templates are segregated into the aqueous compartments of a water/oil emulsion. Under appropriate conditions, huge numbers of aqueous microdroplets can be formed, each containing on average a single DNA template molecule and the machinery of transcription/translation. Because they are surrounded by oil, these compartments do not communicate with one another. The coat polypeptides synthesized in such droplets should associate specifically with the same mRNAs which encode them, and ought to assemble into capsids displaying only one peptide. After synthesis, the emulsion can be broken and the capsids recovered and subjected to selection. In one particular embodiment, all of the transcription/translation reactions are performed in the water/oil emulsion. In one particular embodiment, only droplets containing only one template per droplet (capsids displaying only one peptide) is isolated. In another embodiment, droplets containing mixed capsids may be isolated (plurality of templates per droplet) in one or more cycles of transcription/translation reactions and subsequently capsids displaying only one peptide (one template per droplet) are isolated.

Techniques for affinity selection in phage display are well developed and are directly applicable to the VLP display system of the present invention.

### Self-Assembling Nano-Devices

The VLPs of the present invention may comprise scFv's with affinity for either terminus of a filamentous phage particle that display metal binding proteins. A VLP with affinity for either terminus of a filamentous phage particle would create the possibility of connecting these spheres, as well as their contents, to the ends of filamentous phage nanowires. Alternatively, VLPs with improved ability to self-assemble into these arrays may be produced by displaying scFv's with affinity for a particular surface, or that alter the self-association properties of the VLPs themselves.

VLPs of the invention and/or related compositions may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

### Examples

The invention may be better understood by reference to the following non-limiting example(s), which is provided as exemplary of the invention. The following example is presented in order to more fully illustrate the preferred embodiments of the invention and should in no way be construed, however, as limiting the broad scope of the invention. Reference citations appear after the example.

### Experimental Overview

We displayed scFv's on the VLPs of bacteriophage MS2. The VLPs encapsidated the mRNA that encoded the coat protein from which it was assembled, enabling the recovery by reverse transcription and PCR of affinity selected sequences from scFv libraries.

For proof of concept studies, we utilized three different scFv's:

1. The M18 scFv binds to B. anthracis (anthrax) protective antigen. Both its nucleotide sequence and its three-dimensional structure in complex with anthrax protective antigen have been determined (4). Moreover, anthrax protective antigen is readily available from commercial sources, facilitating tests of M18 function.

2. Anti-AF20 scFv recognizes the AF20 antigen, which is of the overexpressed on hepatocellular carcinoma cells. It provides a convenient model for binding and internalization of VLP-scFv's to cell lines (e.g. Hep3B) that expression the AF20 antigen.

3. CD19 is a surface protein antigen of B cells of the immune system and is present, for example, on B cell leukemia cells. We recreated an scFv version of the CD19-specific HD37 antibody and fused it to VLPs

### Rationale.

As described above, plasmid pDSP62 (see FIG. 2) uses a T7 promoter to express a single-chain dimer coat protein that efficiently assembles into a VLP. To demonstrate the utility of MS2 VLPs for scFv display, we first constructed the plasmid called pDSP62ampst, which differs from pDSP62 by the introduction of an amber (UAG) stop codon in place of the normal stop codons of the wild-type coat protein sequence, and by the introduction of a PstI restriction site. The scFv sequences were synthesized by assembly PCR from synthetic oligonucleotides, using a sequence design optimized for E. coli codon usage. The pDSP62-M18, AF20 and CD19 plasmids were constructed from pDSPampst by cloning the scFV sequences between PstI and BamHI sites. The relevant differences of the pDSP62-scFV's from pDSP62 are as follows:

1. The termination codon of the coat protein single-chain dimer was mutated to the amber stop codon UAG.
2. A synthetic version of the M18, anti-AF20, and anti-CD19 scFv's were cloned downstream of the new UAG, in-frame with the coat sequence.

This arrangement enables display of the scFv's on VLPs, when expressed from The plasmids in cells that also contain pNMsupA, a plasmid that produces an amber suppressor (tRNA). Ribosomes translating the mRNA produced by pDSP62-scFv will normally terminate translation at the UAG, thereby producing only the coat protein single-chain dimer. However, in cells producing the amber suppressor-tRNA form pNMsupA some small percentage of ribosomes will read through the stop codon and proceed through the scFv sequences until encountering its stop codon, roughly an additional 750 nucleotides downstream. This means that two proteins are produced from a single mRNA—large amounts of the single-chain coat protein dimer, and smaller amounts of the same protein with the scFv fused to its C-terminus. The two proteins should co-assemble into a hybrid VLP with scFv molecules displayed on its surface. The average number of scFv's per particle is determined by the nonsense suppression efficiency, but under normal circumstances VLPs will display only a few copies per VLP. The VLP is expected to encapsidate the mRNA that encodes its synthesis.

### Demonstrating Expression of the scFv on MS2 VLPs

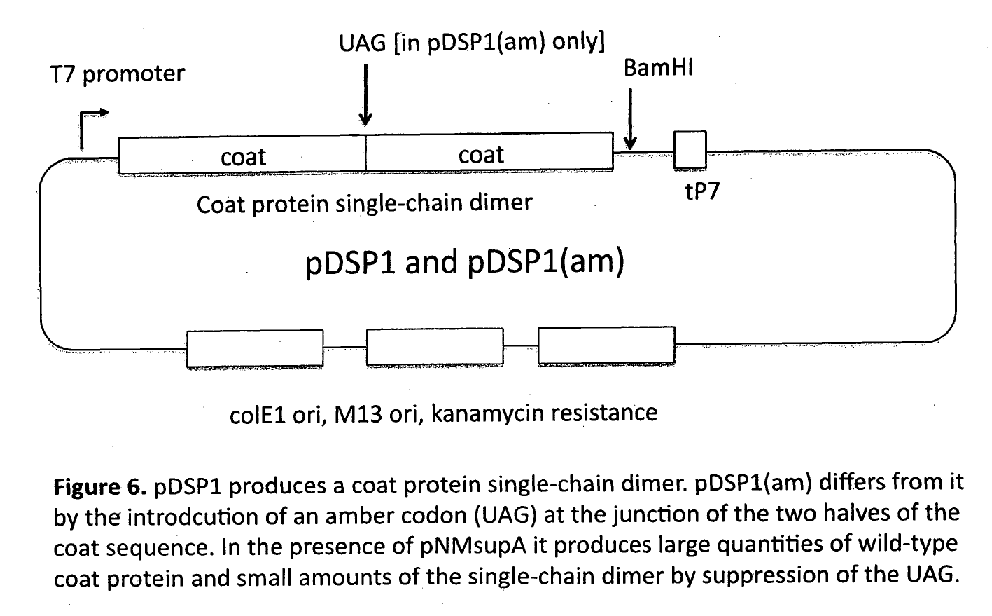

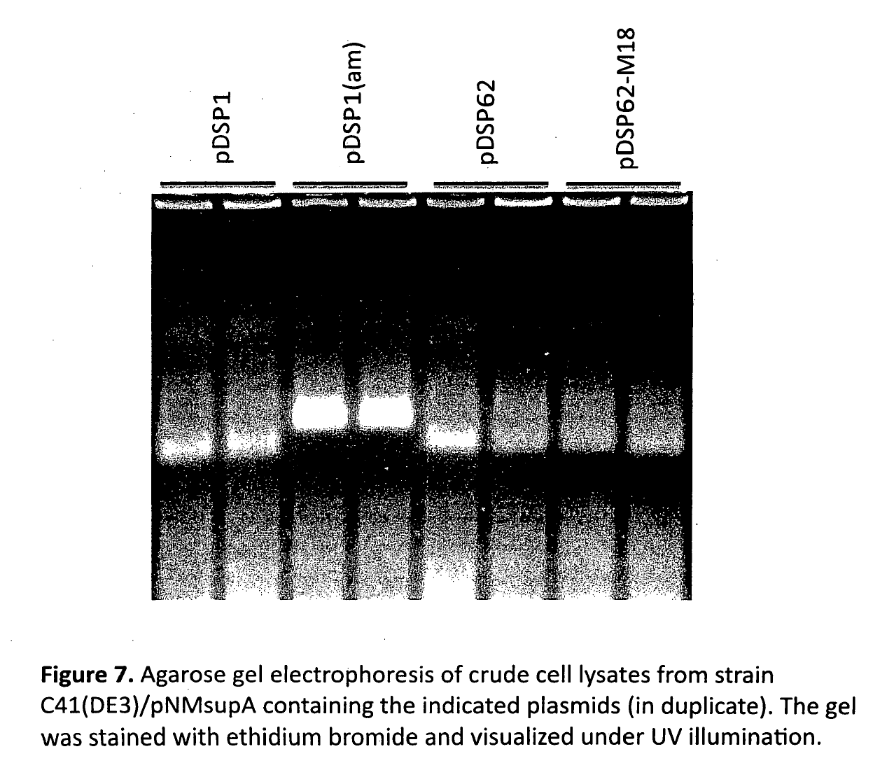

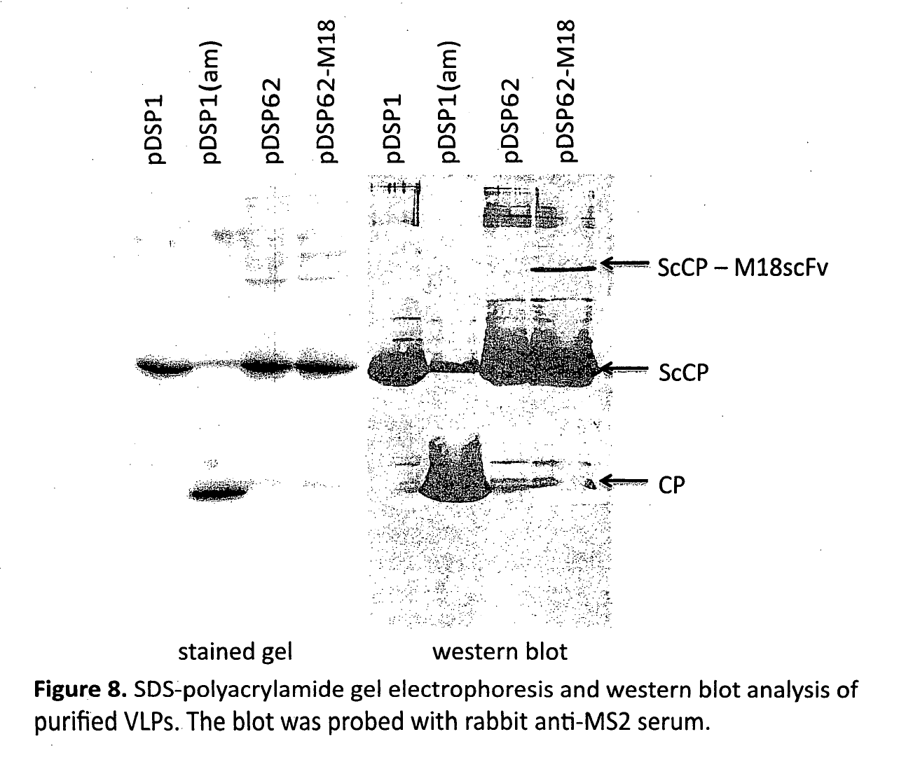

VLP-M18 Expression: Plasmid pDSP61-M18 was introduced into E. coli strain C41(DE3)/pNMsupA where protein and suppressor-tRNA expression were induced with IPTG. As controls we also analyzed the coat proteins synthesized from pDSP1 (FIG. 6) and pDSP62 (FIG. 2), both of which produce single-chain coat protein dimers, and pDSP1 (am) (FIG. 6), which has a UAG codon at the junction between the two halves of the single-chain dimer, and produces wild-type coat protein along with small amounts of the single-chain dimer. Since expression of the single-chain dimer from pDSP1(am) depends on UAG read-through, it serves as an independent measure of nonsense suppression. Agarose gel electrophoresis of cell lysates demonstrated the presence of VLPs in all these samples (FIG. 7). VLPs were then purified by chromatography on Sepharose CL4B and subjected to SDS polyacrylamide gel electrophoresis (FIG. 8). All the control plasmids produce the expected proteins. Meanwhile, pDSP62-M18 produces large amounts of the single-chain dimer, and smaller quantities of a larger protein whose electrophoretic mobility (using coat protein monomer and single-chain dimer as standards) is consistent with that expected for the read-through product (i.e. the M18 scFV fused to the single-chain coat protein dimer). A western blot of the gel shows that the read-through protein is reactive with anti-MS2 serum, as expected.

To determine whether the M18 scFv is folded correctly and is accessible on the particle surface, purified VLPs were bound to anthrax protective antigen previously immobilized in the wells of a plastic microtiter plate. After washing, wells were incubated with polyclonal rabbit anti-MS2 serum and an enzyme-labeled secondary antibody that recognizes rabbit IgG. A chromogenic enzyme substrate was then added and the optical density (OD) determined. This ELISA assay (Table I) shows that VLPs produced by pDSP62-M18 react strongly with protective antigen, and that control wild type VLPs (from pDSP62) do not.

TABLE I
ELISA of wild-type VLPs and M18-displaying VLPs. Well
of a microtiter plate were coated with anthrax protective
antigen, which was then reacted with VLPs. Anti which
was then reacted with VLPs. Anti-MS2 serum and an alkaline
phosphataseconjugated second antibody and chromogenic
substrate were used to detect VLP binding.

| | OD |
|---|---|
| Volume M18 VLP | |
| (ul @5 mg/ml) | |
| 10 | 1.265 |
| 5 | 0.767 |
| Vol. sc-dimer VLP | |
| (ul @5 mg/ml) | |
| 10 | 0.267 |
| 5 | 0.232 |

In summary, these results show that in the presence of an amber suppressor-tRNA, pDSP62-M18 produces a read-through product consisting of a single-chain dimer coat protein with the M18 scFv sequence fused to its C-terminus. The scFv is found at the VLP surface where it is available for recognition of anthrax protective antigen.

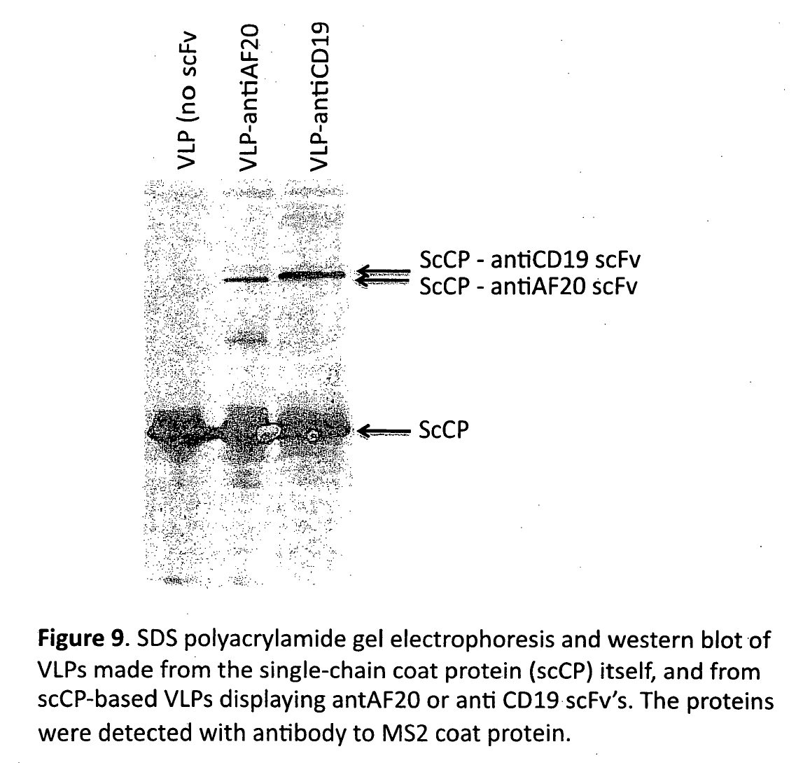

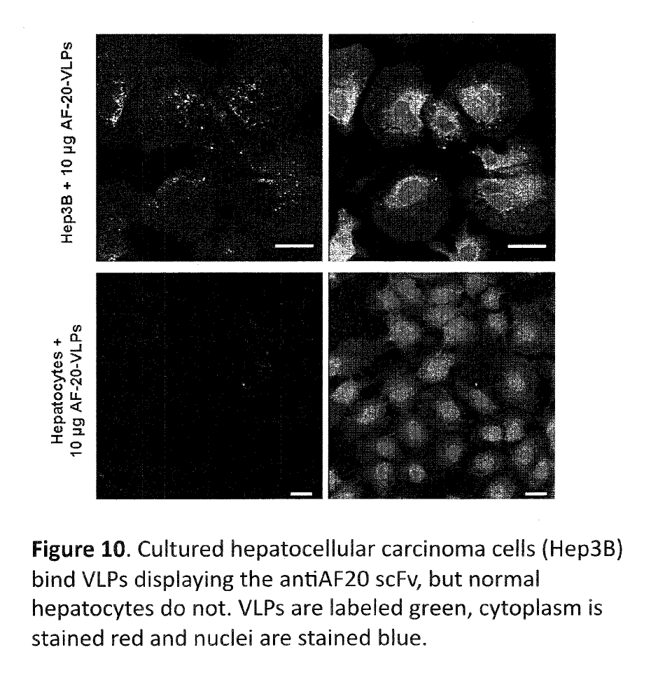

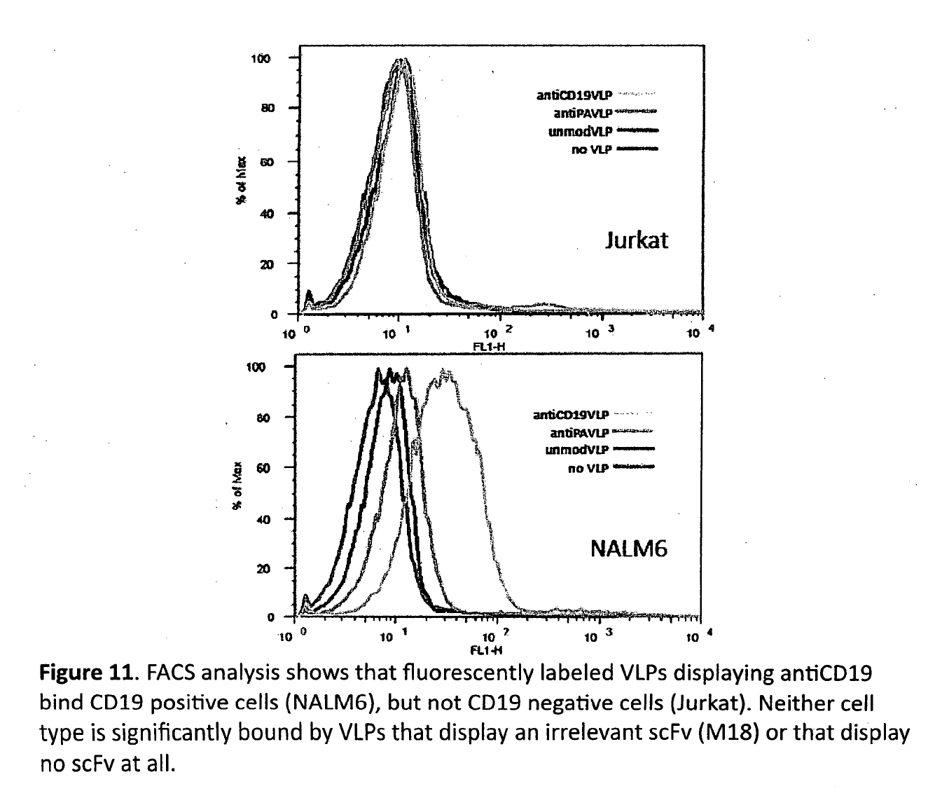

VLP-antiCD19 and VLP-antiAF20 expression: By methods similar to those described above, the pDSP62antiAF20 and pDSPantiCD19 plasmids were expressed in E. coli in the presence of the nonsense suppressor, and the resulting VLPs were purified. FIG. 8 shows a western blot of the VLPs. Each shows the presence of the expected excess of single-chain dimer coat protein, but smaller amounts of the coat-scFv readthrough protein are also present. To determine whether the VLPs could bind their respective antigens, the antiAF20 VLPs were labeled with AlexaFluor488 and bound to Hep3B hepatocellular carcinoma cells and to normal hepatocytes. Fluorescence microscopy (FIG. 9) shows that the particles bind selectively to the Hep3B cells, presumably because they express AF20 on their surfaces. Hepatocytes bind little or no VLP. The anti-CD19 VLPs were similarly labeled and their binding to CD19+ NALM6 cells was determined by Fluorescence Activated Cell Sorting analysis (FACS). In FIG. 10 it is seen that the antiCD19-VLPs bind to the CD19-positive NALM6 cells, but not to Jurkat cells which lack CD19. Moreover, VLPs displaying an irrelevant scFv (M18) or no scFv at all, bind poorly or not at all to either cell type.

### Discussion

In the examples presented here, the scFv is fused to the 3′-end (carboxy terminus) of a coat protein single-chain dimer, but an unduplicated coat sequence would presumably also function for this purpose, and, assuming correct folding and co-assembly, would yield twice the scFv display valency of the single-chain dimer construct.

It should also be noted that when the coat protein and scFv sequences are separated by a stop codon, the level of suppressor-tRNA expression determines the relative amounts of the normally terminated and read-through proteins and, therefore, of the valency of the VLPs produced. Nonsense suppression is normally inefficient (e.g. a few percent), but high-level over-expression of an efficient suppressor can result in perhaps 80% read-through. Note that fusion of the scFv to coat protein through nonsense suppression (rather than direct fusion through a sense codon) is a means of deliberately maintaining a low display valency. Low valency facilitates the selection of high affinity scFv's from libraries, and may also help ensure correct VLP assembly, which might not tolerate a high density of C-terminal fusions. However, it should be recognized that high-density display is an alternative possibility.

The existence of a system for display of scFv's on bacteriophage VLPs means that it is now possible to construct libraries of scFv-presenting particles from which antibody fragments with specific ligand binding activities may be isolated by affinity-selection. The largest scFv libraries now available on filamentous phages have complexities in the 2-3×1010 range, but such libraries are obtained only with tremendous effort. The in vitro MS2 VLP library construction and screening methods described herein enable a more rapid construction of libraries with equal or significantly higher complexities.

Notably, scFv-displaying bacteriophage VLPs should provide new vehicles for targeted drug delivery to specific cell-types (which is useful, for example, in cancer chemotherapy and imaging applications). We have already utilized MS2 VLPs combined with a targeting peptide to delivery drugs cells in a cell type-specific manner. The low valency of scFv display on MS2 VLPs, and the possibility of utilizing single-chain antibodies of human origin, provide a means of avoiding an immune response to the scFv. Also, epitopes on the VLP itself could be masked by chemical modification (e.g. PEG-ylation).

Any polypeptide could potentially be displayed on bacteriophage VLPs using the scheme detailed here. For example, the invention enables the construction of open-reading-frame libraries (from cDNAs) that enable the identification of binding partners by affinity selection against a wide diversity of protein and non-protein targets.

### Additional Details Regarding Plasmid Construction

Plasmid pDSP62 ampst was constructed as a derivative of pDSP62 in which the tandem translation terminators (TAATAG) of the coat sequence were replaced with a single UAG, and a PstI site was introduced between the stop codon and the BamHI site. Between the stop codon and PstI three glycine codons were inserted to serve as a linker between the end of the coat protein and the beginning of the scFv sequence. Here are the sequences of pDSP62 and pDSP62 ampst in the vicinity of the 3′-end of the coat protein coding sequence. Relevant stop codons and restriction sites are in bold.

pDSP62:
...GCAAACTCCGGCATCTACTAATAGACGCCGGGTTAATTAATTAGG
ATCCGGCTGCTA...
pDSP62ampst:
...GCAAACTCCGGCATCTACTAGGGCGGCGGCCTGCAGACGCCGGGT
TAATTAATTAGGATCCGGCTGCTA...

To construct pDSP62-M18, a synthetic M18 sequence was cloned between these PstI and BamHI sites of pDSP62 ampst. The M18 sequence was synthesized by assembly PCR using 25 synthetic oligonucleotides designed using Gene Design software (found at http://baderlab.bme.jhu.edu/gd/index.html). The sequence was designed for optimal E coli codon usage. This generated the following sequence, which was digested with PstI and BamHI and cloned into pDSP62 ampst to produce the plasmid known as pDSP62-M18. The correctness of the resulting M18 sequence was verified by DNA sequence analysis.

Plasmids that direct the synthesis of VLPs displaying anti-AF20 and anti-CD19 scFvs were constructed by similar means incorporating the sequences listed below. Note that in all cases a linker of at least a few amino acids was placed between the C-terminal coat amino acid and the N-terminal scFv amino acid. For example in the case of the antiAF20 recombinant, a total of 8 amino acids, mostly glycines, were included as a linker between the coat and scFv sequences. It is anticipated that especially when the scFv is fused to the C-terminus of coat protein, such linkers may be needed to prevent steric occlusion of the scFv binding site by coat protein itself.

The assembled M18 anti-protective antigen scFv
sequence (SEQ ID NO: 3):
CCCCTGCAGATGGCTGACTACAAAGACATCCAGATGACCCAGACCACC
TCTTCTCTGTCTGCTTCTCTGGGTGACCGTGTTACCGTTTCTTGCCGT
GCTTCTCAGGACATCCGTAACTACCTGAACTGGTACCAGCAGAAACCG
GACGGTACCGTTAAATTCCTGATCTACTACACCTCTCGTCTGCAACCG
GGTGTTCCGTCTCGTTTCTCTGGTTCTGGTTCTGGTACCGACTACTCT
CTGACCATCAACAACCTGGAACAGGAAGACATCGGTACCTACTTCTGC
CAGCAGGGTAACACCCCGCCGTGGACCTTCGGTGGTGGTACCAAACTG
GAAATCAAACGTGGTGGAGGCGGGTCAGGCGGAGGTGGCTCCGGAGGT
GGCGGATCGGGTGGCGGAGGGTCTGAAGTTCAGCTGCAACAGTCTGGT
CCAGAACTGGTTAAACCGGGTGCTTCTGTTAAAATCTCTTGCAAAGAC
TCTGGTTACGCTTTCAACTCTTCTTGGATGAACTGGGTTAAACAGCGT
CCGGGTCAGGGTCTGGAATGGATCGGTCGTATCTACCCGGGTGACGGT
GACTCTAACTACAACGGTAAATTCGAAGGTAAAGCTATCCTGACCGCT
GACAAATCTTCTTCTACCGCTTACATGCAGCTGTCTTCTCTGACCTCT
GTTGACTCTGCTGTTTACTTCTGCGCTCGTTCTGGTCTGCTGCGTTAC
GCTATGGACTACTGGGGTCAGGGTACCTCTGTTACCGTTTCTTCTTAA
GGA
The assembled anti-AF20 scFv sequence
(SEQ ID NO: 4):
CTGCAGGGCGGCGGCCAGCTCCAGCAGTCTGGTCCGGACCTGGTTAAA
CCGGGTGCTTCTGTTCGTATCTCTTGCAAGGCTTCTGGTTACACCTTC
GCTGGTCACTACGTTCACTGGGTTAAACAGCGTCCGGGTCGTGGTCTG
GAATGGATCGGTTGGATCTTCCCGGGTAAAGTTAACACCAAATACAAC
GAAAAATTCAAAGGTAAAGCTACCCTGACCGCTGACAAATCTTCTTCT
ACCGCTTACATGCAGCTGTCTTCTCTGACCTCTGAAGACTCTGCTGTT
TACTTCTGCGCTCGTGTTGGTTACGACTACCCGTACTACTTCGACTAC
TGGGGTCAGGGTACCACCCTGACCGTTTCTTCTGGAGGTGGCGGGTCT
GGGGGCGGTGGATCGGGCGGTGGAGGATCAGGCGGAGGTGGGTCCGAC
ATCCTGCTGACCCAGTCTCCGGCTATCCTGTCTGTTTCTCCGGGTGAC
CGTGTAAGCTTCTCTTGCCGTGCTTCTCAGTCTATCGGTACCTCTATC
CACTGGTACCAGCAGCGTACCAACGGTTCTCCGCGTCTGCTGATCAAA
TACGCTTCTGAATCTATCTCTGGTATCCCGTCTCGTTTCTCTGGTTCT
GGTTCTGGTACCGACTTCACCCTGTCTATCAACTCTGTTGAATCTGAA
GACGTTGCTGACTACTACTGCCAGCAGTCTTCTTCTTGGCCGTTCACC
TTCGGTTCTGGTACCAAACTGGAAATCAAATAAGGATCC
The assembled anti-CD19 scFv sequence
(SEQ ID NO: 5):
CTGCAGGGCGGCGGCCAGGTTCAGCTGCAACAGTCTGGTGCTGAACTG
GTTCGTCCGGGTTCTTCTGTTAAAATCTCTTGCAAAGCTTCTGGTTAC
GCTTTCTCTTCTTACTGGATGAACTGGGTTAAACAGCGTCCGGGTCAG
GGTCTGGAATGGATCGGTCAGATCTGGCCGGGTGACGGTGACACCAAC
TACAACGGTAAATTCAAAGGTAAAGCTACCCTGACCGCTGACGAATCT
TCTTCTACCGCTTACATGCAGCTGTCTTCTCTGGCTTCTGAAGACTCT
GCTGTTTACTTCTGCGCTCGTCGTGAAACCACCACCGTTGGTCGTTAC
TACTACGCTATGGACTACTGGGGTCAGGGTACCTCTGTTACCGTTTCT
TCTGCTGACACCACCCCGAAACTGGAAGAAGGTGAATTCTCTGAAGCT
CGTGTTGACATCCTGCTGACCCAGACCCCGGCTTCTCTGGCTGTTTCT
CTGGGTCAGCGTGCTACCATCTCTTGCAAAGCTTCTCAGTCTGTTGAC
TACGACGGTGACTCTTACCTGAACTGGTACCAGCAGATCCCGGGTCAG
CCGCCGAAACTGCTGATCTACGACGCTTCTAACCTGGTTTCTGGTATC
CCGCCGCGTTTCTCTGGTTCTGGTTCTGGTACCGACTTCACCCTGAAC
ATCCACCCGGTTGAAAAAGTTGACGCTGCTACCTACCACTGCCAGCAG
TCTACCGAAGACCCGTGGACCTTCGGTGGTGGTACCAAACTGGAAATC
AAACGTTGAGGATCC
pDSP62
(SEQ ID NO: 9)
TTCTTAGAAAAACTCATCGAGCATCAAATGAAACTGCAATTTATTCAT
ATCAGGATTATCAATACCATATTTTTGAAAAAGCCGTTTCTGTAATGA
AGGAGAAAACTCACCGAGGCAGTTCCATAGGATGGCAAGATCCTGGTA
TCGGTCTGCGATTCCGACTCGTCCAACATCAATACAACCTATTAATTT
CCCCTCGTCAAAAATAAGGTTATCAAGTGAGAAATCACCATGAGTGAC
GACTGAATCCGGTGAGAATGGCAAAAGCTTATGCATTTCTTTCCAGAC
TTGTTCAACAGGCCAGCCATTACGCTCGTCATCAAAATCACTCGCATC
AACCAAACCGTTATTCATTCGTGATTGCGCCTGAGCGAGACGAAATAC
GCGATCGCTGTTAAAAGGACAATTACAAACAGGAATCGAATGCAACCG
GCGCAGGAACACTGCCAGCGCATCAACAATATTTTCACCTGAATCAGG
ATATTCTTCTAATACCTGGAATGCTGTTTTCCCGGGGATCGCAGTGGT
GAGTAACCATGCATCATCAGGAGTACGGATAAAATGCTTGATGGTCGG
AAGAGGCATAAATTCCGTCAGCCAGTTTAGTCTGACCATCTCATCTGT
AACATCATTGGCAACGCTACCTTTGCCATGTTTCAGAAACAACTCTGG
CGCATCGGGCTTCCCATACAATCGATAGATTGTCGCACCTGATTGCCC
GACATTATCGCGAGCCCATTTATACCCATATAAATCAGCATCCATGTT
GGAATTTAATCGCGGCCTCGAGCAAGACGTTTCCCGTTGAATATGGCT
CATAACACCCCTTGTATTACTGTTTATGTAAGCAGACAGTTTTATTGT
TCATGACCAAAATCCCTTAACGTGAGTTTTCGTTCCACTGAGCGTCAG
ACCCCGTAGAAAAGATCAAAGGATCTTCTTGAGATCCTTTTTTTCTGC
GCGTAATCTGCTGCTTGCAAACAAAAAAACCACCGCTACCAGCGGTGG
TTTGTTTGCCGGATCAAGAGCTACCAACTCTTTTTCCGAAGGTAACTG
GCTTCAGCAGAGCGCAGATACCAAATACTGTCCTTCTAGTGTAGCCGT
AGTTAGGCCACCACTTCAAGAACTCTGTAGCACCGCCTACATACCTCG
CTCTGCTAATCCTGTTACCAGTGGCTGCTGCCAGTGGCGATAAGTCGT
GTCTTACCGGGTTGGACTCAAGACGATAGTTACCGGATAAGGCGCAGC
GGTCGGGCTGAACGGGGGGTTCGTGCACACAGCCCAGCTTGGAGCGAA
CGACCTACACCGAACTGAGATACCTACAGCGTGAGCTATGAGAAAGCG
CCACGCTTCCCGAAGGGAGAAAGGCGGACAGGTATCCGGTAAGCGGCA
GGGTCGGAACAGGAGAGCGCACGAGGGAGCTTCCAGGGGGAAACGCCT
GGTATCTTTATAGTCCTGTCGGGTTTCGCCACCTCTGACTTGAGCGTC
GATTTTTGTGATGCTCGTCAGGGGGGCGGAGCCTATGGAAAAACGCCA
GCAACGCGGCCTTTTTACGGTTCCTGGCCTTTTGCTGGCCTTTTGCTC
ACATGTTCTTTCCTGCGTTATCCCCTGATTCTGTGGATAACCGTATTA
CCGCCTTTGAGTGAGCTGATACCGCTCGCCGCAGCCGAACGACCGAGC
GCAGCGAGTCAGTGAGCGAGGAAGCGGAAGAGCGCCTGATGCGGTATT
TTCTCCTTACGCATCTGTGCGGTATTTCACACCGCATATATGGTGCAC
TCTCAGTACAATCTGCTCTGATGCCGCATAGTTAAGCCAGTATACACT
CCGCTATCGCTACGTGACTGGGTCATGGCTGCGCCCCGACACCCGCCA
ACACCCGCTGACGCGCCCTGACGGGCTTGTCTGCTCCCGGCATCCGCT
TACAGACAAGCTGTGACCGTCTCCGGGAGCTGCATGTGTCAGAGGTTT
TCACCGTCATCACCGAAACGCGCGAGGCAGCTGCGGTAAAGCTCATCA
GCGTGGTCGTGAAGCTTTTCAAAATTGTAAACGTTAATATTTTGTTAA
AATTCGCGTTAAATTTTTGTTAAATCAGCTCATTTTTTAACCAATAGG
CCGAAATCGGCAAAATCCCTTATAAATCAAAAGAATAGCCCGAGATAG
GGTTGAGTGTTGTTCCAGTTTGGAACAAGAGTCCACTATTAAAGAACG
TGGACTCCAACGTCAAAGGGCGAAAAACCGTCTATCAGGGCGATGGCC
CACTACGTGAACCATCACCCAAATCAAGTTTTTGGGGTCGAGGTGCCG
TAAAGCACTAAATCGGAACCCTAAAGGGAGCCCCCGATTTAGAGCTTG
ACGGGGAAAGCCGGCGAACGTGGCGAGAAAGGAAGGGAAGAAAGCGAA
AGGAGCGGGCGCTAGGGCGCTGGCAAGTGTAGCGGTCACGCTGCGCGT
AACCACCACACCCGCCGCGCTTAATGCGCCGCTACAGGGCGCGTACTA
TGGTTGCTTTGACGTCGGCCGCCATGCCGGCGATAATGGCCTGCTTCT
CGCCGAAACGTTTGGTGGCGGGACCAGTGACGAAGGCTTGAGCGAGGG
CGTGCAAGATTCCGAATACCGCAAGCGACAGGCCGATCATCGTCGCGC
TCCAGCGAAAGCGGTCCTCGCCGAAAATGACCCAGAGCGCTGCCGGCA
CCTGTCCTACGAGTTGCATGATAAAGAAGACAGTCATAAGTGCGGCGA
CGATAGTCATGCCCCGCGCCCACCGGAAGGAGCTGACTGGGTTGAAGG
CTCTCAAGGGCATCGGCTCTCCCTTATGCGACTCCTGCATTAGGAAGC
AGCCCAGTAGTAGGTTGAGGCCGTTGAGCACCGCCGCCGCAAGGAATG
GTGCATGCAAGGAGATGGCGCCCAACAGTCCCCCGGCCACGGGGCCTG
CCACCATACCCACGCCGAAACAAGCGCTCATGAGCCCGAAGTGGCGAG
CCCGATCTTCCCCATCGGTGATGTCGGCGATATAGGCGCCAGCAACCG
CACCTGTGGCGCCGGTGATGCCGGCCACGATGCGTCCGGCGTAGAGGA
TCGAGATCTCGATCCCGCGAAATTAATACGACTCACTATAGGGAGACC
ACAACGGTTTCCCTCTAGAAATAATTTTGTTTAACTTTAAGAAGGAGA
TATACCATGGCAAGCAATTTCACGCAATTTGTATTGGTAGATAACGGG
GGTACGGGGGATGTTACGGTAGCACCTTCAAATTTTGCAAATGGTGTA
GCAGAGTGGATATCAAGCAATAGCAGAAGCCAAGCATATAAGGTTACG
TGCTCAGTAAGACAATCAAGCGCTCAAAACAGAAAGTATACGATAAAG
GTAGAAGTTCCGAAGGTTGCTACGCAAACGGTAGGTGGTGTTGAATTG
CCGGTTGCAGCTTGGAGAAGCTATCTCAACATGGAGTTGACGATACCT
ATATTTGCAACCAACAGTGATTGTGAATTGATAGTAAAAGCTATGCAG
GGGTTGTTGAAGGACGGTAATCCTATACCGAGCGCTATAGCTGCTAAT
AGTGGCCTCTACGGCAACTTTACTCAGTTCGTTCTCGTCGACAATGGC
GGAACTGGCGACGTGACTGTCGCCCCAAGCAACTTCGCTAACGGGGTC
GCTGAATGGATCAGCTCTAACTCGCGTTCACAGGCTTACAAAGTAACC
TGTAGCGTTCGTCAGAGCTCTGCGCAGAATCGCAAATACACCATCAAA
GTCGAGGTGCCTAAAGTGGCAACCCAGACTGTTGGTGGTGTAGAGCTT
CCTGTAGCCGCATGGCGTTCGTACTTAAATATGGAACTAACCATTCCA
ATTTTCGCTACGAATTCCGACTGCGAGCTTATTGTTAAGGCAATGCAA
GGTCTCCTAAAAGATGGAAACCCGATTCCCTCAGCAATCGCAGCAAAC
TCCGGCATCTACTAATAGACGCCGGGTTAATTAATTAGGATCCGGCTG
CTAACAAAGCCCGAAAGGAAGCTGAGTTGGCTGCTGCCACCGCTGAGC
AATAACTAGCATAACCCCTTGGGGCCTCTAAACGGGTCTTGAGGGGTT
TTTTGCTGAAAGGAGGAACTATATCCGGATATCCACAGGACGGGTGTG
GTCGCCATGATCGCGTAGTCGATAGTGGCTCCAAGTAGCGAAGCGAGC
AGGACTGGGCGGCGGCCAAAGCGGTCGGACAGTGCTCCGAGAACGGGT
GCGCATAGAAATTGCATCAACGCATATAGCGCTAGCAGCACGCCATAG
TGACTGGCGATGCTGTCGGAATGGACGATATCCCGCAAGAGGCCCGGC
AGTACCGGCATAACCAAGCCTATGCCTACAGCATCCAGGGTGACGGTG
CCGAGGATGACGATGAGCGCATTGTTAGATTTCATACACGGTGCCTGA
CTGCGTTAGCAATTTAACTGTGATAAACTACCGCATTAAAGCTTATCG
ATGATAAGCTGTCAAACATGAA
pDSP62ampst
(SEQ ID NO: 10)
TTCTTAGAAAAACTCATCGAGCATCAAATGAAACTGCAATTTATTCAT
ATCAGGATTATCAATACCATATTTTTGAAAAAGCCGTTTCTGTAATGA
AGGAGAAAACTCACCGAGGCAGTTCCATAGGATGGCAAGATCCTGGTA
TCGGTCTGCGATTCCGACTCGTCCAACATCAATACAACCTATTAATTT
CCCCTCGTCAAAAATAAGGTTATCAAGTGAGAAATCACCATGAGTGAC
GACTGAATCCGGTGAGAATGGCAAAAGCTTATGCATTTCTTTCCAGAC
TTGTTCAACAGGCCAGCCATTACGCTCGTCATCAAAATCACTCGCATC
AACCAAACCGTTATTCATTCGTGATTGCGCCTGAGCGAGACGAAATAC
GCGATCGCTGTTAAAAGGACAATTACAAACAGGAATCGAATGCAACCG
GCGCAGGAACACTGCCAGCGCATCAACAATATTTTCACCTGAATCAGG
ATATTCTTCTAATACCTGGAATGCTGTTTTCCCGGGGATCGCAGTGGT
GAGTAACCATGCATCATCAGGAGTACGGATAAAATGCTTGATGGTCGG
AAGAGGCATAAATTCCGTCAGCCAGTTTAGTCTGACCATCTCATCTGT
AACATCATTGGCAACGCTACCTTTGCCATGTTTCAGAAACAACTCTGG
CGCATCGGGCTTCCCATACAATCGATAGATTGTCGCACCTGATTGCCC
GACATTATCGCGAGCCCATTTATACCCATATAAATCAGCATCCATGTT
GGAATTTAATCGCGGCCTCGAGCAAGACGTTTCCCGTTGAATATGGCT
CATAACACCCCTTGTATTACTGTTTATGTAAGCAGACAGTTTTATTGT
TCATGACCAAAATCCCTTAACGTGAGTTTTCGTTCCACTGAGCGTCAG
ACCCCGTAGAAAAGATCAAAGGATCTTCTTGAGATCCTTTTTTTCTGC
GCGTAATCTGCTGCTTGCAAACAAAAAAACCACCGCTACCAGCGGTGG
TTTGTTTGCCGGATCAAGAGCTACCAACTCTTTTTCCGAAGGTAACTG
GCTTCAGCAGAGCGCAGATACCAAATACTGTCCTTCTAGTGTAGCCGT
AGTTAGGCCACCACTTCAAGAACTCTGTAGCACCGCCTACATACCTCG
CTCTGCTAATCCTGTTACCAGTGGCTGCTGCCAGTGGCGATAAGTCGT
GTCTTACCGGGTTGGACTCAAGACGATAGTTACCGGATAAGGCGCAGC
GGTCGGGCTGAACGGGGGGTTCGTGCACACAGCCCAGCTTGGAGCGAA
CGACCTACACCGAACTGAGATACCTACAGCGTGAGCTATGAGAAAGCG
CCACGCTTCCCGAAGGGAGAAAGGCGGACAGGTATCCGGTAAGCGGCA
GGGTCGGAACAGGAGAGCGCACGAGGGAGCTTCCAGGGGGAAACGCCT
GGTATCTTTATAGTCCTGTCGGGTTTCGCCACCTCTGACTTGAGCGTC
GATTTTTGTGATGCTCGTCAGGGGGGCGGAGCCTATGGAAAAACGCCA
GCAACGCGGCCTTTTTACGGTTCCTGGCCTTTTGCTGGCCTTTTGCTC
ACATGTTCTTTCCTGCGTTATCCCCTGATTCTGTGGATAACCGTATTA
CCGCCTTTGAGTGAGCTGATACCGCTCGCCGCAGCCGAACGACCGAGC
GCAGCGAGTCAGTGAGCGAGGAAGCGGAAGAGCGCCTGATGCGGTATT
TTCTCCTTACGCATCTGTGCGGTATTTCACACCGCATATATGGTGCAC
TCTCAGTACAATCTGCTCTGATGCCGCATAGTTAAGCCAGTATACACT
CCGCTATCGCTACGTGACTGGGTCATGGCTGCGCCCCGACACCCGCCA
ACACCCGCTGACGCGCCCTGACGGGCTTGTCTGCTCCCGGCATCCGCT
TACAGACAAGCTGTGACCGTCTCCGGGAGCTGCATGTGTCAGAGGTTT
TCACCGTCATCACCGAAACGCGCGAGGCAGCTGCGGTAAAGCTCATCA
GCGTGGTCGTGAAGCTTTTCAAAATTGTAAACGTTAATATTTTGTTAA
AATTCGCGTTAAATTTTTGTTAAATCAGCTCATTTTTTAACCAATAGG
CCGAAATCGGCAAAATCCCTTATAAATCAAAAGAATAGCCCGAGATAG
GGTTGAGTGTTGTTCCAGTTTGGAACAAGAGTCCACTATTAAAGAACG
TGGACTCCAACGTCAAAGGGCGAAAAACCGTCTATCAGGGCGATGGCC
CACTACGTGAACCATCACCCAAATCAAGTTTTTGGGGTCGAGGTGCCG
TAAAGCACTAAATCGGAACCCTAAAGGGAGCCCCCGATTTAGAGCTTG
ACGGGGAAAGCCGGCGAACGTGGCGAGAAAGGAAGGGAAGAAAGCGAA
AGGAGCGGGCGCTAGGGCGCTGGCAAGTGTAGCGGTCACGCTGCGCGT
AACCACCACACCCGCCGCGCTTAATGCGCCGCTACAGGGCGCGTACTA
TGGTTGCTTTGACGTCGGCCGCCATGCCGGCGATAATGGCCTGCTTCT
CGCCGAAACGTTTGGTGGCGGGACCAGTGACGAAGGCTTGAGCGAGGG
CGTGCAAGATTCCGAATACCGCAAGCGACAGGCCGATCATCGTCGCGC
TCCAGCGAAAGCGGTCCTCGCCGAAAATGACCCAGAGCGCTGCCGGCA
CCTGTCCTACGAGTTGCATGATAAAGAAGACAGTCATAAGTGCGGCGA
CGATAGTCATGCCCCGCGCCCACCGGAAGGAGCTGACTGGGTTGAAGG
CTCTCAAGGGCATCGGCTCTCCCTTATGCGACTCCTGCATTAGGAAGC
AGCCCAGTAGTAGGTTGAGGCCGTTGAGCACCGCCGCCGCAAGGAATG
GTGCATGCAAGGAGATGGCGCCCAACAGTCCCCCGGCCACGGGGCCTG
CCACCATACCCACGCCGAAACAAGCGCTCATGAGCCCGAAGTGGCGAG
CCCGATCTTCCCCATCGGTGATGTCGGCGATATAGGCGCCAGCAACCG
CACCTGTGGCGCCGGTGATGCCGGCCACGATGCGTCCGGCGTAGAGGA
TCGAGATCTCGATCCCGCGAAATTAATACGACTCACTATAGGGAGACC
ACAACGGTTTCCCTCTAGAAATAATTTTGTTTAACTTTAAGAAGGAGA
TATACCATGGCAAGCAATTTCACGCAATTTGTATTGGTAGATAACGGG
GGTACGGGGGATGTTACGGTAGCACCTTCAAATTTTGCAAATGGTGTA
GCAGAGTGGATATCAAGCAATAGCAGAAGCCAAGCATATAAGGTTACG
TGCTCAGTAAGACAATCAAGCGCTCAAAACAGAAAGTATACGATAAAG
GTAGAAGTTCCGAAGGTTGCTACGCAAACGGTAGGTGGTGTTGAATTG
CCGGTTGCAGCTTGGAGAAGCTATCTCAACATGGAGTTGACGATACCT
ATATTTGCAACCAACAGTGATTGTGAATTGATAGTAAAAGCTATGCAG
GGGTTGTTGAAGGACGGTAATCCTATACCGAGCGCTATAGCTGCTAAT
AGTGGCCTCTACGGCAACTTTACTCAGTTCGTTCTCGTCGACAATGGC
GGAACTGGCGACGTGACTGTCGCCCCAAGCAACTTCGCTAACGGGGTC
GCTGAATGGATCAGCTCTAACTCGCGTTCACAGGCTTACAAAGTAACC
TGTAGCGTTCGTCAGAGCTCTGCGCAGAATCGCAAATACACCATCAAA
GTCGAGGTGCCTAAAGTGGCAACCCAGACTGTTGGTGGTGTAGAGCTT
CCTGTAGCCGCATGGCGTTCGTACTTAAATATGGAACTAACCATTCCA
ATTTTCGCTACGAATTCCGACTGCGAGCTTATTGTTAAGGCAATGCAA
GGTCTCCTAAAAGATGGAAACCCGATTCCCTCAGCAATCGCAGCAAAC
TCCGGCATCTACTAGGGCGGCGGCCTGCAGACGCCGGGTTAATTAATT
AGGATCCGGCTGCTAACAAAGCCCGAAAGGAAGCTGAGTTGGCTGCTG
CCACCGCTGAGCAATAACTAGCATAACCCCTTGGGGCCTCTAAACGGG
TCTTGAGGGGTTTTTTGCTGAAAGGAGGAACTATATCCGGATATCCAC
AGGACGGGTGTGGTCGCCATGATCGCGTAGTCGATAGTGGCTCCAAGT
AGCGAAGCGAGCAGGACTGGGCGGCGGCCAAAGCGGTCGGACAGTGCT
CCGAGAACGGGTGCGCATAGAAATTGCATCAACGCATATAGCGCTAGC
AGCACGCCATAGTGACTGGCGATGCTGTCGGAATGGACGATATCCCGC
AAGAGGCCCGGCAGTACCGGCATAACCAAGCCTATGCCTACAGCATCC
AGGGTGACGGTGCCGAGGATGACGATGAGCGCATTGTTAGATTTCATA
CACGGTGCCTGACTGCGTTAGCAATTTAACTGTGATAAACTACCGCAT
TAAAGCTTATCGATGATAAGCTGTCAAACATGAA

### REFERENCES

1A. Kunkel, T. A., Bebenek, K., and Mcclary, J. (1991) Methods in Enzymology 204, 125-139
2A. Peabody, D. S., and Lim, F. (1996) Nucleic Acids Res 24, 2352-2359
3A. Sidhu, S. S., Lowman, H. B., Cunningham, B. C., and Wells, J. A. (2000) Methods Enzymol 328, 333-363
4A. Chang, A. C., and Cohen, S. N. (1978) J Bacteriol 134, 1141-1156
1. Chang, A. C., and Cohen, S. N. (1978) J Bacteriol 134, 1141-1156
2. Kleina, L. G., Masson, J. M., Normanly, J., Abelson, J., and Miller, J. H. (1990) J Mol Biol 213, 705-717
3. Normanly, J., Kleina, L. G., Masson, J. M., Abelson, J., and Miller, J. H. (1990) J Mol Biol 213, 719-726
4. Leysath, C. E., Monzingo, A. F., Maynard, J. A., Barnett, J., Georgiou, G., Iverson, B. L., and Robertus, J. D. (2009) J Mol Biol 387, 680-693

1. A virus-like particle (VLP) of a RNA bacteriophage comprising an interior core surrounded by a capsid comprising a coat protein of the RNA bacteriophage, wherein a scFv peptide that binds to a target epitope is inserted into the coat protein.

2. The VLP according to claim 1 wherein said scFv peptide binds to a target cell.

3. The VLP of claim 1 wherein said RNA bacteriophage is selected from the group consisting of MS2, Qβ, R17, SP, PP7, GA, M11, MX1, f4, AP205, PRRI, Cb5, Cb12r, Cb23r, 7s and f2.

4. (canceled)

5. (canceled)

6. The VLP of claim 1 wherein said RNA bacteriophage is MS2.

7. The VLP of claim 1, wherein the coat protein is a single chain dimer comprising an upstream and a downstream subunit.

8. The VLP of claim 7, wherein said scFv peptide is inserted at the carboxy end of the downstream subunit.

9. The VLP of claim 7, wherein said scFv peptide is inserted in the AB loop of the downstream subunit.

10. The VLP of claim 1, wherein the interior core comprises one or more bioactive agent or imaging agent.

11. The VLP of claim 10, wherein the bioactive agent is a cytotoxic agent.

12. The VLP of claim 10, wherein the interior core comprises one or more imaging agents.

13. The VLP of claim 11, wherein the one or more cytotoxic agents is an anti-cancer agent, an anti-viral agent, a biologically active RNA, or a toxin.

14. The VLP of claim 13 wherein said biologically active RNA is a small-interfering RNA (shRNA), micro RNA (miRNA) a short hairpin RNA (shRNA) or a mixture thereof.

15. The VLP of claim 1, wherein one or more polypeptides are coupled to the coat protein via a crosslinker molecule.

16. The VLP of claim 13, wherein the anticancer agent is selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

17. (canceled)

18. The VLP according to claim 10 wherein said bioactive agent is an anti-viral agent.

19. (canceled)

20. (canceled)

21. The VLP of claim 10, wherein said bioactive agent is coupled to a nucleic acid molecule that induces formation of the VLP and encapsidation of the bioactive agent within the interior core of the VLP.

22. The VLP of claim 21, wherein the nucleic acid molecule comprises an MS2 pac site.

23. (canceled)

24. The VLP of claim 10, wherein the one or more bioactive agents are coupled to the nucleic acid molecule via a crosslinker molecule.

25. (canceled)

26. (canceled)

27. The VLP of claim 1, wherein the coat protein further comprises a polypeptide that induces osmotic swelling and destabilizes lysosome membranes at a pKa of 6.

28. A method of treating cancer in a patient in need thereof, comprising administering the VLP of claim 13 to the patient in an amount effective to treat the cancer.

29. The method according to claim 28 wherein said cancer is selected from the group consisting of squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

30. A method of killing a cancer cell in a patient, comprising administering the VLP of claim 13 to the patient in an amount sufficient to kill the cancer cell.

31. The VLP of claim 1 further comprising one or more binding peptides other than said scFv peptide.

32. The VLP of claim 2, wherein the target cell is a cancer cell.

33. The VLP of claim 2, wherein the target cell expresses CD99, CD19, CD22, CRLF2, or transferrin receptor.

34. The VLP of claim 1 which exhibits low valency.

35. The VLP of claim 34 which is a mosaic of wild-type coat polypeptide and a single chain dimer coat polypeptide, wherein a scFv peptide that binds to a target epitope is inserted into the single chain dimer coat polypeptide to produce a scFv containing coat protein, said VLP comprising a majority of wild-type coat polypeptide and a minority of scFv containing coat polypeptide.

36. The VLP according to claim 1 wherein said scFv peptide has a selective affinity to a pure recombinant protein, a hapten, a complex antigen, a toxin, an environmental antigen, or a cancer cell-related antigen.

37. A method of determining whether a sample contains a cancer cell, comprising treating the sample with the VLP of claim 12 which binds to a cancer cell, removing unbound VLP from the sample, and determining if the one or more imaging agents are detected in the sample, wherein if the one or more imaging agents are detected in the sample, the sample is identified as containing the cancer cell.

38. A method of determining whether a sample contains a cell which expresses CD99, comprising treating the sample with the VLP of claim 12 which binds to CD99, removing unbound VLP from the sample and determining if one or more imaging agents are detected in the sample, wherein if the one or more imaging agents are detected in the sample, the sample is identified as containing the cell which expresses CD99.

39. A nucleic acid construct comprising:
(a) a bacterial or bacteriophage promoter which is operably associated with a coding sequence of a bacteriophage coat protein or single chain coat polypeptide dimer, wherein the coat polypeptide dimer coding sequence is optionally modified to contain a suppressible nonsense codon;
(b) a nucleotide sequence which encodes an antibody single-chain variable fragment (scFv) and which is in-frame with, and positioned 3′ to, the coat polypeptide dimer coding sequence's termination codon;
(c) a first restriction site positioned 3′ to the coat polypeptide dimer coding sequence and 5′ to the antibody single-chain variable fragment nucleotide sequence and a second restriction site positioned 3′ to the antibody single-chain variable fragment nucleotide sequence;
(d) a PCR primer positioned 3′ to the second restriction site;
(e) a gene for resistance to a first antibiotic; and
(f) a replication origin for replication in a prokaryotic cell, and
Optionally, a second plasmid, in combination with said first plasmid which comprises:
(a) the gene for a nonsense suppressor tRNA to promote translational readthrough of the coat protein stop codon in said first plasmid, thus allowing the synthesis of a coat protein-scFv fusion protein;
(b) a prokaryotic origin of replication; and
(c) a gene for resistance to a second antibiotic.

40. (canceled)

41. (canceled)

42. (canceled)

43. (canceled)

44. (canceled)

45. (canceled)

46. (canceled)

47. (canceled)

48. A nucleic acid sequence of SEQ ID NO:3, SEQ ID NO 4 or SEQ ID NO 5.

49. (canceled)

50. (canceled)

51. A cell, preferably a prokaryote cell, transformed by the nucleic acid construct of claim 39.

52. (canceled)

53. (canceled)

54. (canceled)

55. A method for constructing a library of virus-like particles, the method comprising:
(a) providing a plurality of a nucleic acid constructs comprising
(1) a bacterial or bacteriophage promoter which is operably associated with a coding sequence of a bacteriophage single chain coat polypeptide dimer, wherein the coat polypeptide dimer coding sequence is modified to contain a suppressible stop codon
(2) a first restriction site positioned 3′ to the coat polypeptide dimer coding sequence and 5′ to the antibody single-chain variable fragment nucleotide sequence and a second restriction site positioned 3′ to the antibody single-chain variable fragment nucleotide sequence;
(3) a gene for resistance to a first antibiotic, and
(4) a replication origin for replication in a prokaryotic cell;
(b) treating the nucleic acid constructs with a restriction enzyme;
(c) obtaining a population of transcription units by inserting into the nucleic acid constructs, in a position which is in-frame with and 3′ to the coat polypeptide dimer coding sequence's termination codon, a nucleotide sequence which encodes an antibody single-chain variable fragment; and
(d) expressing the transcription units and, optionally, isolating the library, wherein each particle comprises a bacteriophage coat polypeptide modified by insertion of the antibody single-chain variable fragment, and wherein the antibody single-chain variable fragment is displayed on the virus-like particle and encapsidates bacteriophage mRNA.

56. A method for identifying a scFv, the method comprising:
(a) providing a population of the virus-like particles that have been expressed by a prokaryote which has been transformed by a nucleic acid construct which expresses a bacteriophage coat polypeptide modified by insertion of the antibody single-chain variable fragment, and wherein the antibody single-chain variable fragment is displayed on the virus-like particle and encapsidates bacteriophage mRNA; and
(b) assaying the binding activity of antibody single-chain variable fragments expressed on the virus-like particles.

57. The method of claim 55, wherein the method further comprises:
(a) amplifying those antibody single-chain variable fragments which have been identified as possessing a desired activity by affinity selection; and optionally
(b) isolating the scFv.

58. A diagnostic assay or kit comprising a virus-like particle according to claim 1 which has been expressed by a prokaryote which has been transformed by a nucleic acid construct of claim 39.

59. A method of expressing a library of scFv sequences displayed on VLPs, and affinity-selecting one or more VLP-scFv's with binding activity for a specific target molecule comprising:
1. Constructing a library of scFv's in an expression plasmid;
2. Introducing the library obtained in step 1 into an expression cell where each transformant produces a VLP displaying an scFv with a different ligand specificity;
3. Extracting the VLPs obtained in step 2 from the expression cells;
4. Subjecting the extracted VLPs to affinity selection of a binding target;
5. Washing away and discarding VLPs that fail to bind the binding target during step 4;
6. Eluting VLPs that bind the binding target;
7. Copying the RNA contained within the VLPs eluted from step 6 into DNA by reverse transcription and amplifying said DNA by polymerase chain reaction; and
8. Recloning the amplified DNA obtained from step 7 for production of VLPs, which VLPs are either used in additional rounds of affinity selection, or, if affinity selection is complete, characterizing the selected scFv's with respect to binding affinity and specificity and optionally, determining their sequences.

60.-63. (canceled)
